(19) United States
(12) Patent Application Publication
(10) Pub. No.: US 2026/0366008 A1
(43) Pub. Date: Nov. 19, 2026

(54) EXTRACTION-LESS REVERSE PHASE (RP) CHROMATOGRAPHY FOR MRNA PURITY ASSESSMENT

(71) Applicant: ModernaTX, Inc., Cambridge, MA (US)

(72) Inventors: Emma Abdalla, Cambridge, MA (US); Kevin Cognata, Cambridge, MA (US); Meredith Packer, Cambridge, MA (US)

(73) Assignee: ModernaTX, Inc., Cambridge, MA (US)

(21) Appl. No.: 19/099,965

(22) PCT Filed: Jul. 31, 2023
(86) PCT No.: PCT/US2023/029107

§ 371 Date: Jan. 30, 2025

Related U.S. Application Data
(60) Provisional application No. 63/394,117, filed on Aug. 1, 2022.

(51) Int. Cl. C12N 15/10; A61K 9/51; A61K 31/7105; C12Q 1/68

(52) U.S. Cl. CPC C12N 15/101; A61K 9/5123; A61K 31/7105; C12Q 1/68; C12N 2310/317; C12N 2830/50; C12Q 2600/166

(57) ABSTRACT

Aspects of the disclosure relate to liquid chromatography (e.g., HPLC) methods which enable identification of one or more target nucleic acids in a mixture (e.g., pharmaceutical composition). The disclosure is based, in part, on methods that allow for addition of pharmaceutical compositions (e.g., lipid-based pharmaceutical compositions) directly onto a chromatographic column without the need for first separating target nucleic acids out of the composition. Accordingly, in some embodiments, methods described by the disclosure are useful for assessing the quality of pharmaceutical preparations comprising nucleic acids.

### RELATED APPLICATION

This application claims the benefit under 35 U.S.C § 119(e) of U.S. provisional Application No. 63/394,117, filed Aug. 1, 2022, the contents of which are incorporated by reference herein in their entirety.

### BACKGROUND

HPLC methods utilized for large polynucleotides (such as mRNAs), and pharmaceutical compositions containing such polynucleotides, may have inferior resolution. Furthermore, multiple polyadenylated RNAs formulated in the same mixture (e.g. a mixture of multiple RNAs contained within a lipid-based particle) may be challenging to resolve or coelute.

### SUMMARY

Aspects of the disclosure relate to liquid chromatography (e.g., HPLC) methods which enable identification of one or more target nucleic acids in a mixture (e.g., a pharmaceutical composition) and/or encapsulated in lipid nanoparticles. The disclosure is based, in part, on methods that allow for addition of pharmaceutical compositions (e.g., lipid-based pharmaceutical compositions) directly onto a chromatographic column without the need for first extracting target nucleic acids out of the pharmaceutical composition. Accordingly, in some embodiments, methods described by the disclosure are useful for assessing the quality of pharmaceutical preparations comprising nucleic acids.

Accordingly, the present disclosure provides, in some aspects, a method for identifying a target mRNA, the method comprising:
(i) contacting a stationary phase of a reverse phase chromatography column with one or more mRNAs encapsulated in one or more lipid nanoparticles;
(ii) contacting the column with a mobile phase comprising a first solvent and a second solvent solution, each solvent solution comprising at least one ion pairing agent and at least one inorganic salt, wherein the second solvent solution comprises at least 50% v/v of an organic solvent, such that the target mRNA traverses the column with a retention time that is characteristic of the target mRNA;
(ii) detecting a signal corresponding to the retention time of the target mRNA; and
(iii) identifying the target mRNA as being present based upon detecting the signal corresponding to the retention time of the target mRNA,
wherein the method does not comprise extracting mRNAs from the lipid nanoparticles prior to step (i).

In some embodiments, the at least one inorganic salt in the first and/or second solvent solutions is selected from the group consisting of a sodium salt, potassium salt, lithium salt, magnesium salt, calcium salt, and ammonium salt, optionally wherein the sodium salt is sodium chloride, sodium bromide, sodium acetate, sodium phosphate, or sodium acetate, the potassium salt is potassium chloride, potassium bromide, potassium acetate, potassium phosphate, or potassium acetate, the lithium salt is lithium chloride, lithium bromide, lithium acetate, lithium phosphate, or lithium acetate, the magnesium salt is magnesium chloride, magnesium bromide, magnesium acetate, magnesium phosphate, or magnesium acetate, the calcium salt is calcium chloride, calcium bromide, calcium acetate, calcium phosphate, or calcium acetate, the ammonium salt is ammonium chloride, ammonium bromide, ammonium acetate, ammonium phosphate, or ammonium acetate. In some embodiments, the first and second solvent solutions comprise the same inorganic salt. In some embodiments, the concentration of each of the at least one inorganic salts in the first solvent solution and/or the second solvent solution ranges from about 10 mM-10 M, 20 mM-9 M, 30 mM-8 M, 40 mM-7 M, 50 mM-6 M, 75 mM-5 M, 100 mM-2.5 M, 125 mM-2 M, 150 mM-1.5 M, 175 mM-1 M, or 200 mM-500 mM, optionally wherein the concentration of each of the at least one inorganic salts in the first solvent solution and/or the second solvent solution ranges from about 10 mM-1 M, 40 mM-300 mM, 50 mM-500 mM, 75 mM-400 mM, 100 mM-300 mM, 200-300 mM, 200-250 mM, or 250-300 mM. In some embodiments, each of the first and second solvent solutions comprises the same inorganic salt.

In some embodiments, the first solvent solution and second solvent solution each comprise at least two ion pairing agents in a molar ratio of between about 1:10 to about 10:1. In some embodiments, the first and/or second solvent solution are in a molar ratio between about 1:4 to about 4:1, about 1:5 to about 5:1, about 1:5 to about 5:1, about 1:3 to about 3:1, about 1:2 to about 2:1, or about 1:1.5 to about 1.5:1. In some embodiments, the at least two ion pairing agents in the first and/or second solvent solution are in a 1:1 molar ratio.

In some embodiments, the at least one ion pairing agent in the first and/or second solvent solution is selected from the group consisting of a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt, optionally wherein the trietheylammonium salt is triethylammonium acetate, the tetrabutylammonium salt is tetrabutylammonium phosphate, tetrabutylammonium bromide, or tetrabutylammonium chloride, the hexylammonium salt is hexylammonium acetate or hexylammonium bromide, the dibutylammonium salt is dibutylammonium acetate, the tetrapropylammonium salt is dodecyltrimethylammonium chloride, the tetra(decyl)ammonium salt is tetra(decyl)ammonium bromide, the dihexylammonium salt is dihexylammonium acetate, the dipropylammonium salt is dipropylammonium acetate, the myristyltrimethylammonium salt is myristyltrimethylammonium bromide, the tetraethylammonium salt is tetraethylammonium bromide, the etraheptylammonium salt is tetraheptylammonium bromide, the tetrahexylammonium salt is tetrahexylammonium bromide, the tetrakis(decyl)ammonium salt is tetrakis(decyl)ammonium bromide, the tetramethylammonium salt is tetramethylammonium bromide, the tetraoctylammonium salt is tetraoctylammonium bromide, and/or the tetrapentylammonium salt is tetrapentylammonium bromide.

In some embodiments, the first solvent solution and the second solvent solution each comprise at least two ion pairing agents. In some embodiments, the at least two ion pairing agents are (i) tetrapropylammonium bromide and tetrabutylammonium chloride, (ii) dibutylammonium acetate and triethylammonium acetate, or (iii) tetrabutylammonium phosphate and triethylammonium acetate.

In some embodiments, the concentration of each of the at least one ion pairing agents in the first solvent solution and/or the second solvent solution ranges from about 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, the concentration of each of the at least one ion pairing agents in the first solvent solution and/or the second solvent solution ranges from about 10 mM-1M, 40 mM-300 mM, 50 mM-500 mM, 75 mM-400 mM, 100 mM-300 mM, 200-300 mM, 200-250 mM, or 250-300 mM.

In some embodiments, the first solvent solution and/or the second solvent solution comprises 250 mM tetraproplyammonium bromide and 250 mM tetrabutylammonium chloride.

In some embodiments, each of the first and second solvent solutions comprises a single alkylammonium salt and does not comprise more than one alkylammonium salt.

In some embodiments, the first and second solvent solutions comprise the same single alkylammonium salt.

In some embodiments, the single alkylammonium salt in the first and/or second solvent solutions is selected from the group consisting of a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt, optionally wherein the triethylammonium salt is triethylammonium acetate, the tetrabutylammonium salt is tetrabutylammonium phosphate, tetrabutylammonium bromide, or tetrabutylammonium chloride, the hexylammonium salt is hexylammonium acetate or hexylammonium bromide, the dibutylammonium salt is dibutylammonium acetate, the tetrapropylammonium salt is dodecyltrimethylammonium chloride, the tetra(decyl)ammonium salt is tetra(decyl)ammonium bromide, the dihexylammonium salt is dihexylammonium acetate, the dipropylammonium salt is dipropylammonium acetate, the myristyltrimethylammonium salt is myristyltrimethylammonium bromide, the tetraethylammonium salt is tetraethylammonium bromide, the etraheptylammonium salt is tetraheptylammonium bromide, the tetrahexylammonium salt is tetrahexylammonium bromide, the tetrakis(decyl)ammonium salt is tetrakis(decyl)ammonium bromide, the tetramethylammonium salt is tetramethylammonium bromide, the tetraoctylammonium salt is tetraoctylammonium bromide, and/or the tetrapentylammonium salt is tetrapentylammonium bromide.

In some embodiments, the first and second solvent solutions comprise a single alkylammonium salt selected from the group consisting of tetramethylammonium chloride, tetramethylammonium bromide, triethylammonium acetate, tetrapropylammonium bromide, dipropylammonium acetate, tributylammonium chloride, tetrabutylammonium chloride, tetrabutylammonium bromide, dibutylammonium acetate, and hexylammonium acetate.

In some embodiments, the concentration of the single alkylammonium salt in each of the first and second solvent solutions ranges from about 50 mM-5 M, 100 mM-4 M, 200 mM-3 M, 300 mM-2 M, 400 mM-1M, 400 mM-800 mM, 400 mM-600 mM, or 400 mM-500 mM.

In some embodiments, the single alkylammonium salt is selected from the group consisting of triethylammonium acetate, dipropylammonium acetate, and tetrabutylammonium bromide.

In some embodiments, each of the first and second solvent solutions comprises: (a) 400 mM-1.5 M triethylammonium acetate; (b) 400 mM-1.5 M dipropylammonium acetate; (c) 400 mM-1.5 M tetrabutylammonium bromide; (d) 400 mM-1.5 M tetrabutylammonium phosphate; or (e) 400 mM-1.5 M hexylammonium bromide.

In some embodiments, the second solvent solution comprises about 50% to about 95%, about 55% to about 90%, about 60% to about 85%, about 65% to about 80%, or about 70% v/v to about 75% v/v of the organic solvent. In some embodiments, the second solvent solution comprises about 50%, about 60%, about 70%, about 80%, or about 90% v/v of the organic solvent.

In some embodiments, the organic solvent in the second solvent solution is selected from the group consisting of polar aprotic solvents, C1-4 alkanols, C1-6 alkanediols, and C2-4 alkanoic acids.

In some embodiments, the organic solvent in the second solvent solution is selected from the group consisting of acetonitrile, methanol, ethanol, isopropanol, acetone, propanol, tetrahydrofuran, dimethyl sulfoxide, dimethylformamide, and hexylene glycol. In some embodiments, the organic solvent in the second solvent solution is acetonitrile.

In some embodiments, the column is an analytical column, or a preparative column.

In some embodiments, the stationary phase comprises particles. In some embodiments, the particles are hydrophobic or comprise hydrophobic functional groups. In some embodiments, the particles are porous resin particles.

In some embodiments, the particles have a diameter of about 2 μm-about 10 μm, about 2 μm-about 6 μm, or about 4 μm.

In some embodiments, the particles comprise pores having a diameter of about 500 Å to about 5000 Å, about 800 Å to about 3000 Å, or about 1000 Å to about 2000 Å.

In some embodiments, the target mRNA is RNA or DNA, optionally wherein the target mRNA is single-stranded.

In some embodiments, the target mRNA comprises: (i) 5′ and 3′ UTRs; (ii) a 5′ cap, optionally wherein the 5′ cap is a 7-methylguanosine cap or a 7-methylguanosine group analog; and (iii) a 3′ polyadenosine (polyA) tail.

In some embodiments, the target mRNA is mRNA. In some embodiments, the mRNA is in vitro transcribed (IVT) mRNA.

In some embodiments, the target mRNA has a total length of between about 100 nucleotides and about 10,000 nucleotides, about 100 nucleotides to about 5,000 nucleotides, or about 200 nucleotides to about 4,000 nucleotides.

In some embodiments, the pH of the first solvent solution and/or the second solvent solution is between about pH 6.8 and pH 9. In some embodiments, the pH is about 8.0.

In some embodiments, the column has a temperature from about 70 °C to about 90 °C. In some embodiments, the column has a temperature of about 80 °C.

In some embodiments, the volume percentage of the first solvent solution and volume percentage of the second solvent solution in the mobile phase are each varied from 0% to 100%.

In some embodiments, the ratio of the first solvent solution to the second solvent solution is held constant during elution of the mRNA.

In some embodiments, the ratio of the first solvent solution to the second solvent solution is increased or decreased during elution of the mRNA.

In some embodiments, the concentration of each ion pairing agent in the mobile phase is held constant during elution of the mRNA.

In some embodiments, the concentration of one or more ion pairing agents in the mobile phase is not held constant during elution of the mRNA.

In some embodiments, the eluting is gradient or isocratic with respect to the concentration of the organic solvent.

In some embodiments, the method has a run time of between about 10 minutes and about 30 minutes.

In some embodiments, the target mRNA is present in a composition added to the column in an amount ranging from about 0.05 mg/mL to about 1 mg/mL. In some embodiments, the amount is 0.1 mg/mL.

In some embodiments, the method further comprises repeating steps (i) through (iv) without an intervening step of regenerating the reverse phase chromatography column.

In some embodiments, the method further comprises comparing the retention time of the target mRNA to the retention time of a reference mRNA. In some embodiments, the reference mRNA is an unformulated mRNA. In some embodiments, the comparing step comprises comparing an HPLC chromatogram of the identified mRNA with an HPLC chromatogram of the reference mRNA.

In some embodiments, the method further comprises the step of isolating the target mRNA. In some embodiments, the method is used to determine the potency of the target mRNA.

In some aspects, the present disclosure provides a method of quality control of a pharmaceutical composition comprising a target mRNA, the method comprising:
(i) identifying the target mRNA by any one of the methods provided herein;
(ii) comparing the separated mRNA with a reference mRNA; and
(iii) determining that the pharmaceutical composition comprises the target mRNA based on a comparison of the identified mRNA with the reference mRNA. In some embodiments, the comparing step comprises comparing a HPLC chromatogram of the identified mRNA with a HPLC chromatogram of the reference mRNA.

Each of the limitations of the invention can encompass various embodiments of the invention. It is therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

FIG. 1 shows overlaid RP-IP chromatograms comparing direct inject method performance to extracted for mRNA LNPs in an optimized ion pair system.

FIG. 2 shows overlaid RP-IP chromatograms comparing direct inject method performance to extracted for a multivalent mRNA LNP using an optimized ion pair system.

FIG. 3 shows overlaid RP-IP chromatograms of demonstrating method performance across over 200 injections.

FIG. 4 shows overlaid RP-IP chromatograms depicting the method capability to analyze very dilute formulations.

FIG. 5 shows RP-IP chromatograms of in-process LNP samples depicting the method capability to analyze unstable intermediates.

FIG. 6 shows a typical failure mode with nonoptimized chromatography conditions.

FIGS. 7A-7B show the ability of RP-IP direct injection methods to resolve mRNA-lipid adducts (FIG. 7A) or mRNA (FIG. 7B).

FIGS. 8A-8B show resolution of RP-IP direct injection methods at a range of ion pairing agent concentrations. FIG. 8A shows chromatograms comparing resolution of a 5-mRNA mixture at ion pairing agent concentrations of 200 mM or 500 mM. FIG. 8B shows the USP resolution between peaks at each concentration.

FIGS. 9A-9B show the ability of RP-IP direct injection methods to resolve mRNA in lipid nanoparticle compositions using an alkylammonium salt alone (FIG. 9A), or in combination with an inorganic salt (FIG. 9B).

### DETAILED DESCRIPTION

In some aspects, the disclosure relates to liquid chromatography (e.g., HPLC) methods which enable identification of one or more target nucleic acids in a mixture (e.g., a pharmaceutical composition) and/or lipid nanoparticles. Typically, an HPLC apparatus comprises a reservoir containing a mobile phase, a sample input port, a chromatography column containing the stationary phase, and a detection apparatus. HPLC apparatus and methods for HPLC detection of RNA molecules are generally described, for example in U.S. Pat. No. 8,383,340, the entire contents of which are incorporated herein by reference. In some aspects, the disclosure relates to reversed phase ion pairing HPLC (RP-IP HPLC). Generally, RP-IP HPLC refers to a liquid chromatographic methodology in which retention of analytes on an HPLC column is modulated by addition of an ion pairing agent that alters electrostatic interactions between analytes in a sample (e.g., nucleic acids) and the stationary phase of the chromatography column.

The disclosure is based, in part, on the discovery that inclusion of certain combinations of ion pairing agents and salts in certain molar amounts in the mobile phase allows for separation and/or quantification of one or more target nucleic acids in a pharmaceutical composition (e.g., a lipid-based pharmaceutical composition) without the need for first extracting target nucleic acids out of the pharmaceutical composition. Without wishing to be bound by theory, the ion pairing agents, by modulating the retention of analytes on the chromatographic column, enable the “deformulation” of a pharmaceutical preparation comprising one or more target nucleic acids directly on the chromatography column and make the one or more nucleic acids available for further separation. In some embodiments, the ion pairing agents, alone and with other components of the mobile phase, act as surfactants, disrupting lipid nanoparticle integrity to expose the target nucleic acids(s) shortly after contact of the compositions with the mobile phase. The capacity of a mobile phase to disrupt lipid nanoparticle integrity depends upon the type and amount of ion pairing agents in the mobile phase, but the presence of at least one ion pairing agent containing a surfactant chain, such as an organic salt (e.g., a monoammonium salt, diammonium salt, triammonium salt, or quaternary ammonium salt), and the concentration of ions of the ion pairing agent, are believed to contribute to the disruption of lipid nanoparticle integrity. Moreover, certain combinations of an ion pairing agent (e.g., alkylammonium salt) and an inorganic salt, such as sodium chloride, in a mobile phase also contribute to disruption of lipid nanoparticle integrity, allowing the use of lower concentrations of alkylammonium salts required by direct injection RP-IP chromatography methods. Additionally, other parameters, such as the pH, amount, and type of solvents used in the mobile phase, are believed to contribute to in situ deformulation of lipid nanoparticles containing nucleic acids during chromatography. The methods of the disclosure thus eliminate the need for cumbersome extraction steps prior to conducting liquid chromatography (e.g., HPLC) analyses of nucleic acids formulated in pharmaceutical compositions. The methods of the disclosure have several additional advantages such as (i) increased throughput and efficiency without the need to perform manual sample extractions prior to analysis, (ii) elimination of extraction-related artifacts such as artificially low purity due to processing or low sample recovery, (iii) ability to analyze extremely dilute LNP preparations which are challenging to recover through extraction, (iv) ability to analyze unstable formulation process intermediates, (v) ability to reuse the column for multiple sample runs without having to perform intervening regeneration steps, (vi) ability to capture real-time, “t=0” purity information without delays due to extraction, (vii) allowing the use of at-line process analytical technology (PAT) to increase production efficiency, and/or (viii) allowing the use of two-dimensional (2-D) liquid chromatography (e.g., HPLC) to more finely resolve components of multivalent mixtures.

Accordingly, in some aspects, the disclosure provides a method for identifying a target nucleic acid, the method comprising: (i) contacting a stationary phase of a reverse phase chromatography column with one or more nucleic acids encapsulated in one or more lipid nanoparticles; (ii) contacting the column with a mobile phase comprising a first solvent and a second solvent solution, each solvent solution comprising at least one ion pairing agent and at least one inorganic salt, wherein the second solvent solution comprises at least 50% v/v of an organic solvent, such that the target mRNA traverses the column with a retention time that is characteristic of the target mRNA; (iii) detecting one or more signals indicative of the nucleic acids and if present, lipids, traversing the column; and (iv) identifying a target nucleic acid as being present based upon detecting a signal corresponding to a retention time of the target nucleic acid. In some embodiments, each solvent solution comprises at least two ion pairing agents, e.g., in a molar ratio of between about 1:6 to about 6:1. In some embodiments the nucleic acid is comprised in a pharmaceutical composition. In some embodiments, the pharmaceutical composition is a lipid-based pharmaceutical composition. In some embodiments, the lipid-based pharmaceutical composition comprises one or more nucleic acids encapsulated in one or more lipid nanoparticles.

In some aspects, the efficient resolution and/or clearance of compounds such as lipids present in a pharmaceutical composition is facilitated by the presence of high concentrations of one or more organic solvents in the mobile phase. The mobile phase may comprise one or more solvent solutions (e.g., two or more, three or more, four or more, etc.). One or more of the solvent solutions may comprise one or more organic solvents. In some embodiments, a mobile phase comprises a first solvent solution and a second solvent solution. In some embodiments, the disclosure provides a method for identifying a target nucleic acid in a pharmaceutical composition, the method comprising: (i) contacting a stationary phase of a reverse phase chromatography column with the pharmaceutical composition; (ii) contacting the column with a mobile phase comprising a first solvent solution and a second solvent solution each comprising at least two ion pairing agents in a molar ratio of between about 1:4 to about 4:1, and wherein the second solvent solution further comprises at least about 50% v/v of an organic solvent, such that the target nucleic acid traverses the column with a retention time that is characteristic of the target nucleic acid; (iii) detecting a signal corresponding to the retention time of the target nucleic acid; and (iv) identifying the target nucleic acid as being present in the pharmaceutical composition based upon detecting the signal corresponding to the retention time of the target nucleic acid, wherein the method does not comprise extracting nucleic acids from the pharmaceutical composition prior to step (i). In some embodiments, the pharmaceutical composition is a lipid-based pharmaceutical composition. In some embodiments, the lipid-based pharmaceutical composition comprises one or more nucleic acids encapsulated in one or more lipid nanoparticles.

In some embodiments, one or more solvent solutions (e.g., 1, 2, 3, 4, 5, or more) of the mobile phase comprise a combination of at least two ion pairing agents (e.g., 2, 3, 4, 5, or more). As used herein, an “ion pairing agent” or an “ion pair” refers to an agent (e.g., a small molecule) that functions as a counter ion to a charged (e.g., ionized or ionizable) functional group on an HPLC analyte (e.g., a nucleic acid) and thereby changes the retention time of the analyte as it moves through the stationary phase of an HPLC column. Generally, ion paring agents are classified as cationic ion pairing agents (which interact with negatively charged functional groups) or anionic ion pairing agents (which interact with positively charged functional groups). The terms “ion pairing agent” and “ion pair” further encompass an associated counter-ion (e.g., acetate, phosphate, bicarbonate, bromide, chloride, citrate, nitrate, nitrite, oxide, sulfate and the like, for cationic ion pairing agents, and sodium, calcium, and the like, for anionic ion pairing agents). In some embodiments, one or more ion pairing agents utilized in the methods described by the disclosure is a cationic ion pairing agent. Examples of cationic ion pairing agents include but are not limited to certain protonated or quaternary amines (including e.g., primary, secondary and tertiary amines) and salts thereof, such as a trietheylammonium salt (e.g., triethylammonium acetate (TEAA)), a tetrabutylammonium salt (e.g., tetrabutylammonium phosphate (TBAP) or tetrabutylammonium chloride (TBAC)), a hexylammonium salt (e.g., hexylammonium acetate (HAA)), a dibutylammonium salt (e.g., dibutylammonium acetate (DBAA)), a tetrapropylammonium salt (e.g., tetrapropylammonium bromide (TPAB)), a dodecyltrimethylammonium salt (e.g., dodecyltrimethylammonium chloride (DTMAC)), or a tetra(decyl)ammonium salt (e.g., tetra(decyl)ammonium bromide (TDAB)), a dihexylammonium salt (e.g., dihexylammonium acetate (DHAA)), a dipropylammonium salt (e.g., dipropylammonium acetate (DPAA)), a myristyltrimethylammonium salt (e.g., myristyltrimethylammonium bromide (MTEAB)), a tetraethylammonium salt (e.g., tetraethylammonium bromide (TEAB)), a tetraheptylammonium salt (e.g., tetraheptylammonium bromide (THepAB)), a tetrahexylammonium salt (e.g., tetrahexylammonium bromide (THexAB)), a tetrakis(decyl)ammonium salt (e.g., tetrakis(decyl)ammonium bromide (TrDAB)), a tetramethylammonium salt (e.g., tetramethylammonium bromide (TMAB)), a tetraoctylammonium salt (e.g., tetraoctylammonium bromide (TOAB)), or a tetrapentylammonium salt (e.g., tetrapentylammonium bromide (TPeAB)). In some embodiments, one or more solvent solutions of the mobile phase comprise a combination of two or more ion pairing agents selected from the group consisting of a trietheylammonium salt, tributylammonium salt, tetrabutylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, one or more solvent solutions of the mobile phase comprise a combination of two or more ion pairing agents selected from the group consisting of HAA, TBAP, TPAB, TBAC, DBAA, TEAA, DTMAC, TDAB, DHAA, DPAA MTEAB, TEAB, THepAB, THexAB, TrDAB, TMAB, TOAB, and TPeAB. In some embodiments, one or more solvent solutions of the mobile phase comprise a combination of (i) TPAB and TBAC, (ii) DBAA and TEAA, or (iii) TBAP and TEAA. In some embodiments, one or more solvent solutions of the mobile phase comprise a combination of TPAB and TBAC.

In some embodiments, one or more ion pairing agents utilized in the methods described by the disclosure is a cationic ion pairing agent. In some embodiments, the ion pairing agent is a cationic ion pairing agent. In some embodiments, the ion pairing agent is an alkylammonium salt. In some embodiments, one or more solvent solutions of the mobile phase comprise a salt selected from the group consisting of a trietheylammonium salt, tributylammonium salt, tetrabutylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, one or more solvent solutions of the mobile phase comprise HAA, TBAP, TPAB, TBAC, DBAA, TEAA, DTMAC, TDAB, DHAA, DPAA MTEAB, TEAB, THepAB, THexAB, TrDAB, TMAB, TOAB, TPeABHAA, TBAP, TPAB, TBAC, DBAA, TEAA, DTMAC, or TDAB. In some embodiments, each of one or more solvents of the mobile phase comprises one ion pairing agent. In some embodiments, each of one or more solvents of the mobile phase comprises the same ion pairing agent. In some embodiments, each of one or more solvents of the mobile phase comprises a salt selected from the group consisting of a trietheylammonium salt, tributylammonium salt, tetrabutylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, each of one or more solvents of the mobile phase comprises HAA, TBAP, TPAB, TBAC, DBAA, TEAA, DTMAC, TDAB, DHAA, DPAA MTEAB, TEAB, THepAB, THexAB, TrDAB, TMAB, TOAB, TPeABHAA, TBAP, TPAB, TBAC, DBAA, TEAA, DTMAC, or TDAB. A salt of a cation, as used herein, refers to a composition comprising the cation and an anionic counter ion. For example, a “tetrabutylammonium salt” may refer to tetrabutylammonium phosphate, tetrabutylammonium chloride, tetrabutylammonium bromide, tetrabutylammonium phosphate, or another composition comprising the cation tetrabutylammonium and an anionic counter ion. In some embodiments, the ion pairing agent comprises a cation and an anionic counter ion, wherein the cation is selected from the group consisting of trietheylammonium, tributylammonium, tetrabutylammonium, hexylammonium, dibutylammonium, tetrapropylammonium, dodecyltrimethylammonium, tetra(decyl)ammonium, dihexylammonium, dipropylammonium, myristyltrimethylammonium, tetraethylammonium, tetraheptylammonium, tetrahexylammonium, tetrakis(decyl)ammonium, tetramethylammonium, tetraoctylammonium, and tetrapentylammonium, and the anionic counter ion is selected from the group consisting of a bromide, chloride, phosphate, and acetate.

Protonated and quaternary amine ion pairing agents can be represented by the following formula:

R₄N^{⊕}A^{⊖}
wherein each R independently is hydrogen, optionally substituted aliphatic, optionally substituted heteroaliphatic, optionally substituted aryl or optionally substituted heteroaryl; provided that at least one instance of R is not hydrogen; and A is an anionic counter ion.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups. The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-14 aryl”). The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). Suitable anionic counter ions include, but are not limited to, acetate, trifluoroacetate, phosphate, chloride, bromide hexafluorophosphate, sulfate, methylsulfonate, trifluoromethylsulfonate, 1,1,1,3,3,3-hexafluoro-2-propanol (HFIP), 1,1,1,3,3,3-hexafluoro-2-methyl-2-propanol (HFMIP) and the like.

The term “optionally substituted” refers to being substituted or unsubstituted. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction.

In some embodiments, a solvent solution of the mobile phase (e.g., a first solvent solution or a second solvent solution) comprising at least two ion pairing agents are in a molar ratio of between about 1:1,000 to about 1,000:1, such that the nucleic acids and if present, lipids, traverse the column at different rates. In some embodiments, the at least two ion pairing agents are in a molar ratio between about 1:1,000 to about 1,000:1, 1:900 to about 900:1, 1:800 to about 800:1, 1:700 to about 700:1, 1:600 to about 600:1, 1:500 to about 500:1, 1:400 to about 400:1, about 1:300 to about 300:1, about 1:200 to about 200:1, about 1:100 to about 100:1, about 50:1 to about 1:50, about 40:1 to about 1:40, about 30:1 to about 1:30, about 20:1 to about 1:20, or about 10:1 to about 1:10. In some embodiments, each solvent solution comprises at least two ion pairing agents in a molar ratio of between about 1:100 to about 100:1. In some embodiments, the at least two ion pairing agents are in a molar ratio between about 1:100 to about 100:1, 1:90 to about 90:1, 1:80 to about 80:1, 1:70 to about 70:1, 1:60 to about 60:1, 1:50 to about 50:1, 1:40 to about 40:1, about 1:30 to about 30:1, about 1:20 to about 20:1, about 1:10 to about 10:1, about 5:1 to about 1:5, about 4:1 to about 1:4, about 3:1 to about 1:3, or about 2:1 to about 1:2. In some embodiments, the at least two ion pairing agents are in a 1:1 molar ratio.

In some embodiments, a solvent solution of the mobile phase (e.g., a first solvent solution or a second solvent solution) comprises at least two ion pairing agents that are in a molar ratio of between about 1:6 to about 6:1, such that the nucleic acids and if present, lipids, traverse the column at different rates. In some embodiments, each solvent solution comprises at least two ion pairing agents in a molar ratio of between about 1:4 to about 4:1. In some embodiments, the at least two ion pairing agents are in a molar ratio between about 1:3 to about 3:1, about 1:2 to about 2:1, or about 1:1.5 to about 1.5:1. In some embodiments, the at least two ion pairing agents are in a 1:1 molar ratio.

The concentration of each ion pairing agent in a solvent solution (e.g., a first solvent solution or a second solvent solution) may range from about 1 mM to about 25 M (e.g., about 1 mM, about 2 mM, about 5 mM, about 10 mM, about 50 mM, about 100 mM, about 200 mM, about 500 mM, about 1 M, about 1.2 M, about 1.5 M, about 1.75 M, about 2M, about 2.25 M, about 2.5 M, about 2.75 M, about 3 M, about 3.25 M, about 3.5 M, about 3.75 M, about 4 M, about 4.25 M, about 4.5 M, about 4.75 M, about 5 M, about 5.5 M, about 6 M, about 6.5 M, about 7 M, about 7.5 M, about 8 M, about 8.5 M, about 9 M, about 9.5 M, about 10 M, about 11 M, about 12 M, about 13 M, about 14 M, about 15 M, about 16 M, about 17 M, about 18 M, about 19 M, or about 20 M), inclusive. In some embodiments, the concentration of an ion pairing agent in a mobile phase (e.g., a first solvent solution or a second solvent solution) ranges from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, the concentration of each of the ion pairing agents independently ranges from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, a first or second solvent solution comprises a single ion pairing agent, which is present in an amount from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM.

The concentration of each ion pairing agent in a solvent solution (e.g., a first solvent solution or a second solvent solution) may range from about 1 mM to about 2 M (e.g., about 1 mM, about 2 mM, about 5 mM, about 10 mM, about 50 mM, about 100 mM, about 200 mM, about 500 mM, about 1 M, about 1.2 M, about 1.5 M, or about 2M), inclusive. In some embodiments, the concentration of an ion pairing agent in a mobile phase (e.g., a first solvent solution or a second solvent solution) ranges from about, 10 mM-1M, 40 mM-300 mM, 50 mM-500 mM, 75 mM-400 mM, 100 mM-300 mM, 200-300 mM, 200-250 mM, or 250-300 mM. In some embodiments, the concentration of each of the ion pairing agents independently ranges from about, 10 mM-1M, 40 mM-300 mM, 50 mM-500 mM, 75 mM-400 mM, 100 mM-300 mM, 200-300 mM, 200-250 mM, or 250-300 mM. In some embodiments, two ion pairing agents are present at concentrations of about 20 mM:40 mM, 50 mM:50 mM, 50 mM: 60 mM, 50 mM:75 mM, 50 mM:100 mM, 50 mM:150 mM, 100 mM:100 mM, 100 mM:125 mM, 100 mM:150 mM, 100 mM:175 mM, 100 mM:200 mM, 100 mM:200 mM, 100 mM: 250 mM, 100 mM:300 mM, 125 mM:125 mM, 125 mM:150 mM, 125 mM:175 mM, 125 mM:200 mM, 125 mM:250 mM, 125 mM:300 mM, 150 mM:175 mM, 150 mM:200 mM, 150 mM:250 mM, 150 mM:300 mM, 200 mM:200 mM, 200 mM:250 mM, 200 mM:300 mM, 250 mM:250 mM, 250 mM:300 mM, or 300 mM:300 mM.

Examples of ion pairing agent concentrations include but are not limited to 40 mM TEAA: 20 mM DBAA, 100 mM TEAA: 50 mM DBAA, 50 mM TBAP: 50 mM TEAA, 250 mM TBAP: 250 mM TEAA, 300 mM TBAP: 300 mM TEAA, 50 mM TBAP: 150 mM TEAA, 125 mM TBAP: 250 mM TEAA, 250 mM TBAP: 250 mM TEAA, 300 mM TBAP: 300 mM TEAA, 50 mM DBAA: 50 mM TEAA, 60 mM DBAA: 50 mM TEAA, 75 mM DBAA: 50 mM TEAA, 175 mM DBAA: 125 mM TEAA, 100 mM DBAA: 100 mM TEAA, 50 mM TBAP: 100 mM TEAA, 100 mM TBAP: 200 mM TEAA, 125 mM TBAP: 250 mM TEAA, 150 mM TABP: 200 mM TEAA, 150 mM TBAP: 200 mM TEAA, 150 mM TBAP: 250 mM TEAA, 50 mM TBAP: 150 mM TEAA, 100 mM TBAP: 150 mM TEAA, 250 mM TBAP: 200 mM TEAA, 250 mM TBAP: 250 mM TEAA, or 200 mM TBAP: 300 mM TEAA. In some embodiments, one or more solvent solutions of the mobile phase comprise a combination of TPAB and TBAC. In some embodiments, the concentrations of TPAB and TBAC independently range from 50 mM-300 mM. In some embodiments, one or more solvent solutions of the mobile phase comprise 200 mM TPAB: 200 mM TBAC, 250 mM TPAB: 250 mM TBAC, or 300 mM TPAB: 300 mM TBAC. In some embodiments, one or more solvent solutions of the mobile phase comprise 250 mM TPAB: 250 mM TBAC.

In some embodiments, one or more solvent solutions (e.g., 1, 2, 3, 4, 5, or more) of the mobile phase comprise a single ion pairing agent. In some embodiments, one or more solvent solutions comprise no more than one ion pairing agent. In some embodiments, one or more solvent solutions comprise one, and only one, ion pairing agent. In some embodiments, one or more solvent solutions comprise a single ion pairing agent. In some embodiments, each solvent solution comprises the same ion pairing agent. In some embodiments, each solvent solution comprises a single alkylammonium salt. In some embodiments, each solvent solution comprises the same alkylammonium salt. In some embodiments, each of the solvent solutions of a mobile phase comprise a single ion pairing agent.

The concentration of the single ion pairing agent (e.g., alkylammonium salt) in a solvent solution (e.g., a first solvent solution or a second solvent solution) may range from about 1 mM to about 25 M (e.g., about 1 mM, about 2 mM, about 5 mM, about 10 mM, about 50 mM, about 100 mM, about 200 mM, about 500 mM, about 1 M, about 1.2 M, about 1.5 M, about 1.75 M, about 2M, about 2.25 M, about 2.5 M, about 2.75 M, about 3 M, about 3.25 M, about 3.5 M, about 3.75 M, about 4 M, about 4.25 M, about 4.5 M, about 4.75 M, about 5 M, about 5.5 M, about 6 M, about 6.5 M, about 7 M, about 7.5 M, about 8 M, about 8.5 M, about 9 M, about 9.5 M, about 10 M, about 11 M, about 12 M, about 13 M, about 14 M, about 15 M, about 16 M, about 17 M, about 18 M, about 19 M, or about 20 M), inclusive. In some embodiments, the concentration of a single ion pairing agent in a mobile phase (e.g., a first solvent solution or a second solvent solution) ranges from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, the concentration of the single ion pairing agent in each solvent solution independently ranges from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, the concentration of the single ion pairing agent in each solvent solution ranges independently from about 50 mM-5 mM, 100 mM-4 M, 200 mM-3 M, 400 mM-1 M, 400 mM-800 mM, 400 mM-700 mM, 400 mM-600 mM, or 400 mM. In some embodiments, the concentration of the single ion pairing agent in each of the solvent solutions is about 50 mM-5 mM, 100 mM-4 M, 200 mM-3 M, 400 mM-1 M, 400 mM-800 mM, 400 mM-700 mM, 400 mM-600 mM, or 400 mM. In some embodiments, the single ion pairing agent is present in each solvent solution at the same concentration. In some embodiments, the concentration of the single ion pairing agent in the first solvent solution is between 80% and 120%, 90% and 110%, or 95% to 105% of the concentration of the single ion pairing agent in the second solvent solution.

The concentration of the single ion pairing agent in a solvent solution (e.g., a first solvent solution or a second solvent solution) may range from about 1 mM to about 2 M (e.g., about 1 mM, about 2 mM, about 5 mM, about 10 mM, about 50 mM, about 100 mM, about 200 mM, about 400 mM, about 500 mM, about 600 mM, about 700 mM, about 800 mM, about 900 mM, about 1 M, about 1.2 M, about 1.5 M, or about 2M), inclusive. In some embodiments, the concentration of an ion pairing agent in a mobile phase (e.g., a first solvent solution or a second solvent solution) ranges from about 200 mM-400 mM, 400-600 mM, 600 mM-800 mM, 800 mM-1 M, 300 mM-600 mM, 600 mM-900 mM, 400-800 mM, or 400 mM-1 M. In some embodiments, the concentration of the single ion pairing agent in each solvent solution is selected from the group consisting of 200 mM-400 mM, 400-600 mM, 600 mM-800 mM, 800 mM-1 M, 300 mM-600 mM, 600 mM-900 mM, 400-800 mM, or 400 mM-1 M.

In some embodiments, the mobile phase comprises a single alkylammonium salt selected from triethylammonium acetate, dipropylammonium acetate, and tetrabutylammonium bromide. In some embodiments, each solvent solution in a mobile phase comprises the same single alkylammonium salt, and the single alkylammonium salt is selected from triethylammonium acetate, dipropylammonium acetate, and tetrabutylammonium bromide. In some embodiments, the mobile phase comprises triethylammonium acetate at a concentration of 400 mM-1 M. In some embodiments, the mobile phase comprises about 400 mM, 500 mM, 600 mM, 700 mM, 800 mM, 900 mM, or 1 M triethylammonium acetate. In some embodiments, the mobile phase comprises about 400 mM-600 mM, 600 mM-800 mM, or 800 mM-1 M triethylammonium acetate.

In some embodiments, the mobile phase comprises dipropylammonium acetate at a concentration of 400 mM-1 M. In some embodiments, the mobile phase comprises about 400 mM, 500 mM, 600 mM, 700 mM, 800 mM, 900 mM, or 1 M dipropylammonium acetate. In some embodiments, the mobile phase comprises about 400 mM-600 mM, 600 mM-800 mM, or 800 mM-1 M dipropylammonium acetate.

In some embodiments, the mobile phase comprises tetrabutylammonium bromide at a concentration of 400 mM-1 M. In some embodiments, the mobile phase comprises about 400 mM, 500 mM, 600 mM, 700 mM, 800 mM, 900 mM, or 1 M tetrabutylammonium bromide. In some embodiments, the mobile phase comprises about 400 mM-600 mM, 600 mM-800 mM, or 800 mM-1 M tetrabutylammonium bromide.

The concentration of inorganic salt(s) in a solvent solution (e.g., a first solvent solution or a second solvent solution) may range from about 10 mM to about 10 M (e.g., about 10 mM, about 20 mM, about 30 mM, about 40 mM, about 50 mM, about 75 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1 M, about 1.2 M, about 1.5 M, about 1.75 M, about 2M, about 2.25 M, about 2.5 M, about 2.75 M, about 3 M, about 3.25 M, about 3.5 M, about 3.75 M, about 4 M, about 4.25 M, about 4.5 M, about 4.75 M, about 5 M, about 5.5 M, about 6 M, about 6.5 M, about 7 M, about 7.5 M, about 8 M, about 8.5 M, about 9 M, about 9.5 M, or about 10), inclusive. In some embodiments, the concentration of inorganic salt(s) in a mobile phase (e.g., a first solvent solution or a second solvent solution) ranges from 10 mM-10 M, 20 mM-9 M, 30 mM-8 M, 40 mM-7 M, 50 mM-6 M, 75 mM-5 M, 100 mM-2.5 M, 125 mM-2 M, 150 mM-1.5 M, 175 mM-1 M, or 200 mM-500 mM. In some embodiments, the concentration of each inorganic salt in a mobile phase independently ranges from about, 10 mM-10 M, 20 mM-9 M, 30 mM-8 M, 40 mM-7 M, 50 mM-6 M, 75 mM-5 M, 100 mM-2.5 M, 125 mM-2 M, 150 mM-1.5 M, 175 mM-1 M, or 200 mM-500 mM. In some embodiments, a first or second solvent solution comprises a single inorganic salt, which is present in an amount from about, 10 mM-10 M, 20 mM-9 M, 30 mM-8 M, 40 mM-7 M, 50 mM-6 M, 75 mM-5 M, 100 mM-2.5 M, 125 mM-2 M, 150 mM-1.5 M, 175 mM-1 M, or 200 mM-500 mM. In some embodiments, each of the first and second solvent solutions comprises the same inorganic salt, which is present in an amount from, about 10 mM-10 M, 20 mM-9 M, 30 mM-8 M, 40 mM-7 M, 50 mM-6 M, 75 mM-5 M, 100 mM-2.5 M, 125 mM-2 M, 150 mM-1.5 M, 175 mM-1 M, or 200 mM-500 mM. In some embodiments, each of the first and second solvent solutions comprises about 10 mM-10 M, 20 mM-9 M, 30 mM-8 M, 40 mM-7 M, 50 mM-6 M, 75 mM-5 M, 100 mM-2.5 M, 125 mM-2 M, 150 mM-1.5 M, 175 mM-1 M, or 200 mM-500 mM sodium chloride.

The inorganic salt in a solvent solution (e.g., a first solvent solution or a second solvent solution) may be selected from any inorganic salt known in the art. As used herein, an “inorganic salt” refers to a salt that does not comprise a carbon-hydrogen (C—H) bond. Non-limiting examples of inorganic salts include salts containing sodium salts, potassium salts, lithium salts, magnesium salts, calcium salts, and ammonium salts that lack an alkyl chain. In some embodiments, each of the first and/or second solvent solution comprises a sodium salt, lithium salt, potassium salt, magnesium salt, calcium salt, or ammonium salt. In some embodiments, the sodium salt is sodium chloride, sodium bromide, sodium acetate, sodium phosphate, or sodium sulfate. In some embodiments, the sodium salt is sodium chloride. In some embodiments, the sodium salt is sodium bromide. In some embodiments, the sodium salt is sodium acetate. In some embodiments, the sodium salt is sodium phosphate. In some embodiments, the sodium salt is sodium sulfate. In some embodiments, the potassium salt is potassium chloride, potassium bromide, potassium acetate, potassium phosphate, or potassium sulfate. In some embodiments, the potassium salt is potassium chloride. In some embodiments, the potassium salt is potassium bromide. In some embodiments, the potassium salt is potassium acetate. In some embodiments, the potassium salt is potassium phosphate. In some embodiments, the potassium salt is potassium sulfate. In some embodiments, the lithium salt is lithium chloride, lithium bromide, lithium acetate, lithium phosphate, or lithium sulfate. In some embodiments, the lithium salt is lithium chloride. In some embodiments, the lithium salt is lithium bromide. In some embodiments, the lithium salt is lithium acetate. In some embodiments, the lithium salt is lithium phosphate. In some embodiments, the lithium salt is lithium sulfate. In some embodiments, the magnesium salt is magnesium chloride, magnesium bromide, magnesium acetate, magnesium phosphate, or magnesium sulfate. In some embodiments, the magnesium salt is magnesium chloride. In some embodiments, the magnesium salt is magnesium bromide. In some embodiments, the magnesium salt is magnesium acetate. In some embodiments, the magnesium salt is magnesium phosphate. In some embodiments, the magnesium salt is magnesium sulfate. In some embodiments, the calcium salt is calcium chloride, calcium bromide, calcium acetate, calcium phosphate, or calcium sulfate. In some embodiments, the calcium salt is calcium chloride. In some embodiments, the calcium salt is calcium bromide. In some embodiments, the calcium salt is calcium acetate. In some embodiments, the calcium salt is calcium phosphate. In some embodiments, the calcium salt is calcium sulfate. In some embodiments, the ammonium salt is ammonium chloride, ammonium bromide, ammonium acetate, ammonium phosphate, or ammonium sulfate. In some embodiments, the ammonium salt is ammonium chloride. In some embodiments, the ammonium salt is ammonium bromide. In some embodiments, the ammonium salt is ammonium acetate. In some embodiments, the ammonium salt is ammonium phosphate. In some embodiments, the ammonium salt is ammonium sulfate. In some embodiments, a solvent solution (e.g., first and/or second solvent solution) comprising an ion pairing agent and inorganic salt comprises a lower concentration of the ion pairing agent than a solvent solution that does not comprise an inorganic salt. In some embodiments, a mobile phase comprising solvent solutions comprising an ion pairing agent and inorganic salt is capable of deformulating a lipid nanoparticle to the same or a greater extent than a mobile phase comprising a higher amount of the same ion pairing agent, but lacking the inorganic salt. In some embodiments, the mobile phase comprises one or more solvent solutions that each comprise (i) an inorganic salt, and (ii) an ion pairing agent in an amount that is 95% or less, 90% or less, 80% or less, 70% or less, 60% or less, 50% or less, 40% or less, 30% or less, or lower, of the concentration of the ion pairing agent in a comparator mobile phase comprising the same ion pairing agent but lacking an inorganic salt, where the comparator mobile phase is capable of deformulating a lipid nanoparticle to the same or a lesser extent than the mobile phase comprising solvent solutions comprising the inorganic salt and a lower concentration of the same ion pairing agent.

Ion pairing agents and/or inorganic salts are generally dispersed within a mobile phase. As used herein, a “mobile phase” is an aqueous solution comprising water and/or one or more organic solvents used to carry an HPLC analyte (or analytes), such as a nucleic acid encapsulated in a lipid nanoparticle, mixture of nucleic acids in lipid nanoparticles, or a pharmaceutical composition comprising a nucleic acid or mixture of nucleic acids in lipid nanoparticles, through an HPLC column. In some embodiments, a mobile phase for use in HPLC methods as described by the disclosure is comprised of multiple (e.g., 2, 3, 4, 5, or more) solvent solutions. In some embodiments of the HPLC methods described by the disclosure, the mobile phase comprises two solvent solutions, a first solvent solution and a second solvent solution (e.g., Mobile Phase A, and Mobile Phase B). In some embodiments, a solvent solution comprises at least two ion pairing agents in a molar ratio of 1:1,000 to 1,000:1. In some embodiments, each solvent solution (e.g., the first solvent solution and the second solvent solution) comprises at least two ion pairing agents in a molar ratio of 1:1,000 to 1,000:1. In some embodiments, a solvent solution comprises at least two ion pairing agents in a molar ratio of 1:100 to 100:1. In some embodiments, each solvent solution (e.g., the first solvent solution and the second solvent solution) comprises at least two ion pairing agents in a molar ratio of 1:100 to 100:1. In some embodiments, a solvent solution comprises at least two ion pairing agents in a molar ratio of 1:75 to 75:1. In some embodiments, each solvent solution (e.g., the first solvent solution and the second solvent solution) comprises at least two ion pairing agents in a molar ratio of 1:75 to 75:1. In some embodiments, a solvent solution comprises at least two ion pairing agents in a molar ratio of 1:50 to 50:1. In some embodiments, each solvent solution (e.g., the first solvent solution and the second solvent solution) comprises at least two ion pairing agents in a molar ratio of 1:50 to 50:1. In some embodiments, a solvent solution comprises at least two ion pairing agents in a molar ratio of 1:25 to 25:1. In some embodiments, each solvent solution (e.g., the first solvent solution and the second solvent solution) comprises at least two ion pairing agents in a molar ratio of 1:25 to 25:1. In some embodiments, a solvent solution comprises at least two ion pairing agents in a molar ratio of 1:10 to 10:1. In some embodiments, each solvent solution (e.g., the first solvent solution and the second solvent solution) comprises at least two ion pairing agents in a molar ratio of 1:10 to 10:1. In some embodiments, a solvent solution comprises at least two ion pairing agents in a molar ratio of 1:6 to 6:1. In some embodiments, each solvent solution (e.g., the first solvent solution and the second solvent solution) comprises at least two ion pairing agents in a molar ratio of 1:6 to 6:1. In some embodiments, a solvent solution comprises at least two ion pairing agents in a molar ratio of 1:4 to 4:1. In some embodiments, each solvent solution (e.g., the first solvent solution and the second solvent solution) comprises at least two ion pairing agents in a molar ratio of 1:4 to 4:1.

In some embodiments, at least one solvent solution of the mobile phase comprises an organic solvent. Generally, an IP-RP HPLC mobile phase comprises a polar organic solvent. Examples of polar organic solvents suitable for inclusion in a mobile phase include but are not limited to alcohols, ketones, nitrates, esters, amides and alkylsulfoxides. In some embodiments, the mobile phase (e.g., at least one solvent solution of the mobile phase) comprises one or more organic solvents selected from the group consisting of polar aprotic solvents, C₁₋₄ alkanols, C₁₋₆ alkanediols, and C₂₋₄ alkanoic acids. In some embodiments, the mobile phase (e.g., at least one solvent solution of the mobile phase) comprises one or more organic solvents selected form the group consisting of acetone, acetonitrile, dimethylformamide, dimethylsulfoxide (DMSO), ethanol, hexylene glycol, isopropanol, methanol, methyl acetate, propanol, and tetrahydrofuran. In some embodiments, the mobile phase (e.g., at least one solvent solution of the mobile phase) comprises acetonitrile. In some embodiments, a mobile phase (e.g., at least one solvent solution of the mobile phase) comprises additional components, for example as described in U.S. Patent Publication US 2005/0011836, the entire contents of which is incorporated herein by reference.

The concentration of organic solvent in a mobile phase (e.g., each solvent solution of the mobile phase) can vary. For example, in some embodiments, the volume percentage (v/v) of an organic solvent in a mobile phase varies from 0% (absent) to about 100% of a mobile phase. In some embodiments, the volume percentage of organic solvent in a mobile phase (e.g., at least one solvent solution of the mobile phase) is between about 5% and about 75% v/v. In some embodiments, the volume percentage of organic solvent in a mobile phase (e.g., at least one solvent solution of the mobile phase) is between about 25% and about 60% v/v. In some embodiments, the volume percentage of organic solvent in a mobile phase (e.g., at least one solvent solution of the mobile phase) is at least about 50% v/v. In some embodiments, the volume percentage of organic solvent in a mobile phase (e.g., at least one solvent solution of the mobile phase) is about 50% to about 95%, about 55% to about 90%, about 60% to about 85%, about 65% to about 80%, or about 70% v/v to about 75% v/v. In some embodiments, the concentration of organic solvent in a mobile phase (e.g., at least one solvent solution of the mobile phase) is about 5%, about 10%, about 15%, about 20%, about 25%, about 30%, about 35%, about 40%, about 45%, about 50%, about 55%, about 60%, about 65%, about 70%, about 75%, about 80%, about 85%, about 90% v/v, or about 95% v/v.

In some embodiments, the first solvent solution does not comprise an organic solvent. In some embodiments, the volume percentage of organic solvent in the second solvent solution is at least about 50% v/v. In some embodiments, the volume percentage of organic solvent in the second solvent solution is about 50% to about 95%, about 55% to about 90%, about 60% to about 85%, about 65% to about 80%, or about 70% v/v to about 75% v/v. In some embodiments, the volume percentage of organic solvent in the second solvent solution is about 50%, about 55%, about 60%, about 65%, about 70%, about 75%, about 80%, about 85%, about 90% v/v, or about 95% v/v.

The pH of the mobile phase (e.g., the pH of each solvent solution of the mobile phase) can vary. In some embodiments, the pH of the mobile phase is between about pH 5.0 and pH 9.5 (e.g., about 5.0, about 5.5, about 6.0, about 6.5, about 7.0, about 7.5, about 8.0, about 8.5, about 9.0, or about 9.5). In some embodiments, the pH of the mobile phase is between about pH 6.8 and pH 9.0 (e.g., about 6.8, about 7.0, about 7.2, about 7.4, about 7.6, about 7.8, about 8.0, about 8.3, about 8.5, or about 9.0). In some embodiments, the pH of the mobile phase is about 8.0.

In some embodiments, the pH of the first solvent solution is between about pH 5.0 and pH 9.5 (e.g., about 5.0, about 5.5, about 6.0, about 6.5, about 7.0, about 7.5, about 8.0, about 8.5, about 9.0, or about 9.5). In some embodiments, the pH of the first solvent solution is between about pH 6.8 and pH 9.0 (e.g., about 6.8, about 7.0, about 7.2, about 7.4, about 7.6, about 7.8, about 8.0, about 8.3, about 8.5, or about 9.0). In some embodiments, the pH of the first solvent solution is about 8.0.

In some embodiments, the pH of the second solvent solution is between about pH 5.0 and pH 9.5 (e.g., about 5.0, about 5.5, about 6.0, about 6.5, about 7.0, about 7.5, about 8.0, about 8.5, about 9.0, or about 9.5). In some embodiments, the pH of the second solvent solution is between about pH 6.8 and pH 9.0 (e.g., about 6.8, about 7.0, about 7.2, about 7.4, about 7.6, about 7.8, about 8.0, about 8.3, or about 8.5). In some embodiments, the pH of the second solvent solution is about 8.0.

The concentration of two or more solvent solutions in a mobile phase can vary. For example, in a mobile phase comprising two solvent solutions (e.g., a first solvent solution and a second solvent solution), the volume percentage of the first solvent solution may range from about 0% (absent) to about 100%. In some embodiments, the volume percentage of the first solvent solution may range from about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% v/v.

Conversely, in some embodiments, the volume percentage of the second solvent solution of a mobile phase may range from about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% v/v.

In some embodiments, the ratio of the first solvent solution to the second solvent solution is held constant (e.g., isocratic) during elution of the nucleic acid. However, the skilled artisan will appreciate that in other embodiments, the relative ratio of the first solvent solution to the second solvent solution can vary throughout the elution step. For example, in some embodiments, the ratio of the first solvent solution is increased relative to the second solvent solution during the elution step. In some embodiments, the ratio of the first solvent solution is decreased relative to the second solvent solution during the elution step.

The concentration of one or more ion pairing agents in a mobile phase (e.g., a solvent solution) can vary. The relative ratios of the at least two ion pairing agents in a mobile phase (or solvent solution) may vary or be held constant (e.g., isocratic) during the eluting step. In some embodiments, the ratio of a first ion pairing agent is increased relative to a second ion pairing agent during the elution step. In some embodiments, the ratio of a first ion pairing agent is increased relative to a second ion pairing agent during the elution step. For example, in some embodiments, the ratio of TPAB to TBAC ranges from about 4:1 to about 1:4, about 3:1 to about 1:3, about 2:1 to about 1:2, or about 1:1 to 1:3.

The mobile phase (e.g., a solvent solution) may be gradient or isocratic with respect to the concentration of one or more organic solvents.

Any suitable HPLC column (e.g., stationary phase) may be used in the methods described by the disclosure. Generally, a “HPLC column” is a solid structure or support that contains a medium (e.g. a stationary phase) through which the mobile phase and HPLC sample (e.g., a sample containing HPLC analytes, such as nucleic acids) is eluted. Without wishing to be bound by any particular theory, the composition and chemical properties of the stationary phase determine the retention time of HPLC analytes. In some embodiments of HPLC methods described by the disclosure, the stationary phase is non-polar. Examples of non-polar stationary phases include but are not limited to resin, silica (e.g., alkylated and non-alkylated silica), polystyrenes (e.g., alkylated and non-alkylated polystyrenes), polystyrene divinylbenzenes, etc. In some embodiments, a stationary phase comprises particles, for example porous particles. In some embodiments, a stationary phase (e.g., particles of a stationary phase) is hydrophobic (e.g., made of an intrinsically hydrophobic material, such as polystyrene divinylbenzene), or comprise hydrophobic functional groups. In some embodiments, a stationary phase is a membrane or monolithic stationary phase.

The particle size (e.g., as measured by the diameter of the particle) of an HPLC stationary phase can vary. In some embodiments, the particle size of a HPLC stationary phase ranges from about 1 μm to about 100 μm (e.g., any value between 1 and 100, inclusive) in diameter. In some embodiments, the particle size of a HPLC stationary phase ranges from about 2 μm to about 10 μm, about 2 μm to about 6 μm, or about 4 μm in diameter. The pore size of particles (e.g., as measured by the diameter of the pore) can also vary. In some embodiments, the particles comprise pores having a diameter of about 100 Å to about 10,000 Å. In some embodiments, the particles comprise pores having a diameter of about 100 Å to about 5000 Å, about 100 Å to about 1000 Å, or about 1000 Å to about 2000 Å. In some embodiments, the stationary phase comprises polystyrene divinylbenzene, for example as used in PLRP-S 4000 columns or DNAPac-RP columns.

A sample being added to the stationary phase (e.g., a pharmaceutical preparation) may be diluted in a surfactant. Surfactants may include, but are not limited to, one or more of Triton, polysorbate 20, 40, 60, and 80, sodium lauryl sulfate, etc. In some embodiments, the percentage of the surfactant ranges from about 1% to 5%, or about 5% to 10%. In some embodiments, the percentage of the surfactant is about 1%, 2%, 3%, 4%, 5%, 6%, 7%, 8%, 9%, or 10%. In some embodiments, the sample being added to the stationary phase is diluted in the first solvent solution (e.g., Mobile phase A).

In some embodiments, the injection volumes of the sample (e.g., a pharmaceutical preparation) range from about 10 μL to about 100 μL, about 10 μL to about 50 μL, about 20 μL to about 50 μL, about 20 μL to about 70 μL, or about 50 μL to about 100 μL.

The methods of the disclosure allow the use of small doses of pharmaceutical preparations. Accordingly, dose preparation amounts of a pharmaceutical composition may be tested using the methods of the disclosure. In some embodiments, a target nucleic acid is present in the pharmaceutical composition (e.g., lipid-based pharmaceutical composition) in an amount ranging from about 0.05 mg/mL to about 1 mg/mL (e.g., 0.05, 006, 0.07, 0.08, 0.09, 0.1, 0.2. 0.3. 0.4, 0.5, 0.6, 0.7, 0.8, 0.9, or 1 mg/mL). In some embodiments, the target nucleic acid is present in the pharmaceutical composition at about 0.1 mg/mL.

The temperature of the column (e.g., the stationary phase within the column) can vary. In some embodiments, the column has a temperature from about 20 °C to about 99 °C (e.g., any temperature between 20 °C and 99 °C. In some embodiments, the column has a temperature from about 40 °C to about 99 °C (e.g., any temperature between 40 °C and 99 °C, for example about 40 °C, about 50 °C, about 60 °C, about 70 °C, about 80 °C, about 90 °C, about 95 °C, or about 99 °C). In some embodiments, the column has a temperature from about 60 °C to about 90 °C (e.g., any temperature between 60 °C and 90 °C). In some embodiments, the column has a temperature of about 80 °C.

The methods of the disclosure surprisingly allow the column to be used more than once to analyze pharmaceutical compositions (e.g., lipid-based pharmaceutical compositions) comprising one or more nucleic acids without an intervening regeneration step. In some embodiments, the column may be used to run 2-10, 10-20, 20-50, 50-100, 100-200 or more samples without regenerating the column.

In some embodiments, the methods of the disclosure include a wash step. The wash solution may comprise one or more solvents selected from methanol, acetonitrile, tetrahydrofuran, isopropanol, methylene chloride, hexane, ethyl acetate, acetic acid, trifluoroacetic acid, propanol, DMSO, etc.

In some embodiments, HPLC methods as described by the disclosure comprise the step of detecting or isolating a nucleic acid. Any detection apparatus or modality suitable for HPLC may be used. Examples of HPLC detectors include but are not limited to absorbance detectors (e.g., UV/VIS detectors), fluorescence detectors, electrochemical detectors, and mass spectrometric detectors.

In some aspects, the disclosure relates to improved HPLC methods for detection and characterization of one or more nucleic acids in pharmaceutical preparations by directly adding the pharmaceutical composition to an HPLC column, without first extracting the nucleic acids out of the pharmaceutical composition. In some embodiments, the nucleic acids in the pharmaceutical composition are encapsulated in microparticles or nanoparticles. In some embodiments, the pharmaceutical composition is a lipid-based pharmaceutical composition. In some embodiments, the nucleic acids in the pharmaceutical composition are encapsulated in lipid nanoparticles. In some embodiments, a pharmaceutical composition comprises a mixture of two or more nucleic acids (e.g., a bivalent or multivalent composition comprising 2, 3, 4, 5, 6, 7, 8, 9, 10, or more nucleic acids).

As used herein, a “polynucleotide” or “nucleic acid” is at least two nucleotides covalently linked together, and in some instances, may contain phosphodiester bonds (e.g., a phosphodiester “backbone”) or modified bonds, such as phosphorothioate bonds. An “engineered nucleic acid” is a nucleic acid that does not occur in nature. In some instances, the nucleic acid is an engineered nucleic acid. It should be understood, however, that while an engineered nucleic acid as a whole is not naturally-occurring, it may include nucleotide sequences that occur in nature. Thus, a “polynucleotide” or “nucleic acid” includes a series of nucleotide bases (also called “nucleotides”), generally in DNA and RNA. The terms include genomic DNA, cDNA, RNA, any synthetic and genetically manipulated polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone.

Some embodiments involve the analysis of pharmaceutical preparations (e.g., a lipid-based pharmaceutical composition) comprising one or more nucleic acids, for example DNA or RNA. An RNA typically is composed of repeating ribonucleosides. It is possible that the RNA includes one or more deoxyribonucleosides. In preferred embodiments the RNA is comprised of greater than 60%, 70%, 80% or 90% of ribonucleosides. In some embodiments the RNA is 100% comprised of ribonucleosides. The RNA in a mixture is preferably an mRNA.

As used herein, the term “messenger RNA (mRNA)” refers to a ribonucleic acid that has been transcribed from a DNA sequence by an RNA polymerase enzyme, and interacts with a ribosome to synthesize protein encoded by DNA. Generally, mRNA are classified into two sub-classes: pre-mRNA and mature mRNA.

mRNA can be isolated from tissues or cells by a variety of methods. For example, a total RNA extraction can be performed on cells or a cell lysate and the resulting extracted total RNA can be purified (e.g., on a column comprising oligo-dT beads) to obtain extracted mRNA.

Alternatively, mRNA can be synthesized in a cell-free environment, for example by in vitro transcription (IVT). IVT is a process that permits template-directed synthesis of ribonucleic acid (RNA) (e.g., messenger RNA (mRNA)). It is based, generally, on the engineering of a template that includes a bacteriophage promoter sequence upstream of the sequence of interest, followed by transcription using a corresponding RNA polymerase. In vitro mRNA transcripts, for example, may be used as therapeutics in vivo to direct ribosomes to express protein therapeutics within targeted tissues.

Traditionally, the basic components of an mRNA molecule include at least a coding region, a 5′UTR, a 3′UTR, a 5′ cap and a poly-A tail. IVT mRNA may function as mRNA but are distinguished from wild-type mRNA in their functional and/or structural design features which serve to overcome existing problems of effective polypeptide production using nucleic-acid based therapeutics. For example, IVT mRNA may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

A nucleic acid molecule (e.g., DNA or RNA) may comprise naturally occurring nucleotides and/or non-naturally occurring nucleotides such as modified nucleotides. In some embodiments, one or more nucleotides of a polynucleotide includes at least one chemical modification. In some embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. Other exemplary chemical modifications useful in the mRNA described herein include those listed in US Published patent application 2015/0064235, which is incorporated by reference herein in its entirety.

An “in vitro transcription (IVT) template,” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

A “5′ untranslated region (UTR)” refers to a region of an mRNA that is directly upstream (i.e., 5′) from the start codon (i.e., the first codon of an mRNA transcript translated by a ribosome) that does not encode a protein or peptide. In some embodiments, a 5′ UTR encodes a 7-methylguanosine cap or a 7-methylguanosine group analog (e.g., a cap analog for example as described by Kowalska et al. RNA. 2008 June; 14(6): 1119-1131).

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

An “open reading frame” is a continuous stretch of DNA beginning with a start codon (e.g., methionine (ATG)), and ending with a stop codon (e.g., TAA, TAG or TGA) and encodes a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In some embodiments, a polyA tail contains up to 1000 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

In some embodiments, a nucleic acid is larger than 100 nucleotides in length. In some embodiments, a nucleic acid is between about 100 and about 10000 nucleotides in length, about 200 and about 7500 nucleotides in length, or about 500 and about 5000 nucleotides in length.

In some embodiments of methods described by the disclosure, a nucleic acid (e.g., RNA or DNA) is a therapeutic or prophylactic nucleic acid. As used herein, the term “therapeutic nucleic acid” refers to a nucleic acid molecule (e.g., RNA or DNA) that encodes a therapeutic peptide or protein. In some embodiments, an mRNA (e.g., IVT mRNA) is a therapeutic and/or prophylactic mRNA. As used herein, the term “therapeutic mRNA” refers to an mRNA molecule (e.g., an IVT mRNA) that encodes a therapeutic peptide or protein. Therapeutic peptides or proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). As used herein, the term “prophylactic nucleic acid” refers to a nucleic acid molecule (e.g., RNA or DNA) that encodes a prophylactic peptide or protein such a vaccine antigen. As used herein, the term “prophylactic mRNA” refers to an mRNA molecule (e.g., an IVT mRNA) that encodes a prophylactic peptide or protein such as a vaccine antigen. Prophylactic proteins mediate a variety of effects in a host cell or a subject in order to prevent disease. Therapeutic and/or prophylactic nucleic acids (e.g., RNA (e.g., mRNA) or DNA) may be useful for the treatment of the following diseases and conditions: bacterial infections, viral infections, parasitic infections, cell proliferation disorders, genetic disorders, and autoimmune disorders.

Delivery of mRNA molecules to a subject in a therapeutic context is promising because it enables intracellular translation of the mRNA and production of at least one encoded peptide or polypeptide of interest without the need for nucleic acid-based delivery systems (e.g., viral vectors and DNA-based plasmids). Therapeutic mRNA molecules are generally synthesized in a laboratory (e.g., by in vitro transcription). However, there is a potential risk of carrying over impurities or contaminants, such as incorrectly synthesized mRNA, lipid adducts, and/or undesirable synthesis reagents, into the final therapeutic preparation during the production process. In order to prevent the administration of impure or contaminated mRNA, the mRNA molecules can be subject to a quality control (QC) procedure (e.g., validated or identified) prior to use. Validation confirms that the correct mRNA molecule has been synthesized and is pure.

Certain aspects of the disclosure relate to the discovery that HPLC methods described herein are useful, in some embodiments, for quality control of pharmaceutical compositions comprising one or more nucleic acid molecules (e.g., RNA (e.g., mRNA) or DNA).

Accordingly, in some aspects, the disclosure provides a method of quality control of a pharmaceutical composition (e.g., a lipid-based pharmaceutical composition) comprising one or more nucleic acids, the method comprising: separating a target nucleic acid from a pharmaceutical composition by a method as described by the disclosure; comparing the separated nucleic acid with a reference nucleic acid (e.g., a reference unformulated nucleic acid); and determining the quality of the nucleic acid based on a comparison of the separated nucleic acid with the reference nucleic acid.

In some embodiments, the methods of the disclosure include analyzing a pharmaceutical composition comprising a nucleic acid first followed by analyzing the pure, unformulated nucleic acid.

In some instances, the methods of the disclosure are used to determine the stability or integrity of a nucleic acid in a pharmaceutical composition. In some instances, the methods of the disclosure are used to determine the purity of a nucleic acid in a pharmaceutical composition. The term “pure” as used herein refers to material that has only the target nucleic acid active agents such that the presence of unrelated nucleic acids is reduced or eliminated, i.e., impurities or contaminants, including nucleic acid fragments and lipid adducts. Impurities measured by methods described in the disclosure may include nucleic acids that are distinct from a target mRNA (e.g., mRNA fragments generated by mRNA cleavage, mRNAs having different than expected lengths, such as mRNAs lacking poly(A) tails, and contaminating DNA fragments used in in vitro transcription). Impurities may also include lipid adducts formed by covalent addition of lipid species to nucleobases of nucleic acids, which may consequently inhibit translation compared to non-lipidated mRNAs. See, e.g., Packer et al., Nat Commun. 2021. 12(1):6777. Lipid adducts may be formed by bonding between an mRNA or mRNA fragment, and any lipid present in a composition, such as an ionizable amino lipid, non-cationic lipid, structural lipid (e.g., sterol), and/or a PEG-modified lipid. In some embodiments, a purified RNA sample includes one or more target or test nucleic acids but is preferably substantially free of other nucleic acids and lipid adducts. As used herein, the term “substantially free” is used operationally, in the context of analytical testing of the material. Preferably, purified material substantially free of impurities or contaminants is at least 95% pure; more preferably, at least 98% pure, and more preferably still at least 99% pure. In some embodiments a pure nucleic acid sample is comprised of 100% of the target or test nucleic acids and includes no other nucleic acids. In some embodiments, it only includes a single type of target or test nucleic acid. In some embodiments a pure RNA sample is comprised of 100% of the target or test RNAs and includes no other RNA. In some embodiments it only includes a single type of target or test RNA.

A “reference nucleic acid” as used herein refers to a control nucleic acid (e.g. an unformulated nucleic acid) or chromatogram generated from a control nucleic acid that uniquely identifies the nucleic acid separated from the mixture or lipid nanoparticle. The reference nucleic acid may be generated based on digestion of a pure sample and compared to data generated by HPLC of a pharmaceutical composition comprising the nucleic acid of interest. Alternatively it may be a known chromatogram, stored in a electronic or non-electronic data medium. For example, a control chromatogram may be a chromatogram based on predicted HPLC retention times of a particular RNA (e.g., a test mRNA). In some embodiments quality control methods described by the disclosure further comprise the step of comparing the nucleic acid separated from the mixture to the reference nucleic acid using an orthogonal analytical technique, for example polymerase chain reaction (e.g., RT-qPCR), nucleic acid sequencing, gel electrophoresis, mass spectrometry, etc.

In some embodiments, quality control of a pharmaceutical composition comprises (i) subjecting a first portion of the composition to a process that reduces the purity of mRNA in the composition (e.g., by promoting degradation of target mRNA), (ii) using a method described by the disclosure to separately analyze both the first portion, and a second portion of the composition that was not subjected to the purity-reducing process, and (iii) comparing the chromatograms obtained by analyzing each portion. Reduced purity of the composition, as measured by target mRNA amount or concentration, indicates that the method is capable of detecting impurities introduced by processes that are known to reduce mRNA purity or introduce impurities. In some embodiments, the method further comprises, after subjecting the first portion to a purity-reducing process, separating nucleic acids from each of the first and second portions, analyzing the nucleic acids by chromatography, and comparing the resulting chromatograms. In some embodiments, the method comprises measuring the amount of target mRNA degraded and/or the amount of mRNA fragments generated by the purity-reducing process. In some embodiments, the measured change in target mRNA and/or mRNA fragment amounts measured by adding the composition portions directly to columns is 80%-120%, 90%-110%, 95%-105%, 97%-103%, 98%-102%, or 99%-101% of the corresponding change measured by separating nucleic acids from the composition portions before adding the separated nucleic acids to the column. For example, if subjecting a first composition portion to heat stress to promote cleavage of target mRNAs to produce mRNA fragments, such a purity-reducing process may reduce the concentration of target mRNA in the composition, compared to a second portion of the composition that was not subjected to heat stress. If analyzing both portions by a method described by the disclosure indicates that heat stress reduces target mRNA amount in the first composition portion by 0.985 mg/mL compared to the second composition portion, and a method that comprises separating nucleic acids from the heat-stressed composition portions before adding the nucleic acids to columns indicates that heat stress reduces target mRNA abundance by 1.00 mg/mL, then the method described by the disclosure measures a change in target mRNA amounts that is 98.5% of the change measured by a method in which nucleic acids are separated from the composition before analysis. In some embodiments, a purity-reducing process is heat stress. Heat stressing may comprise heating a composition (or portion of a composition) to a temperature of 30 °C or higher, 35 °C or higher, 37 °C or higher, 40 °C or higher, 45 °C or higher, 50 °C or higher, 55 °C or higher, 56 °C or higher, 60 °C or higher, 65 °C or higher, 70 °C or higher, 75 °C or higher, 80 °C or higher, 85 °C or higher, 90 °C or higher, 95 °C or higher, or up to 100 °C. In some embodiments, the composition or portion is heated for 1-60 minutes, 2-50 minutes, 3-45 minutes, 4-30 minutes, 5-20 minutes, 10-15 minutes, 1-5 minutes, 5-10 minutes, 10-15 minutes, 15-20 minutes, 20-30 minutes, 30-45 minutes, 45-60 minutes, 60-90 minutes, 90-120 minutes, 120-180 minutes, or longer.

In some embodiments, a purity-reducing process comprises increasing or lowering the pH of a composition (or portion of a composition). Subjecting a composition to a lower or higher pH may be used to promote acidic or basic hydrolysis, respectively. of nucleic acids. In some embodiments, the pH is lowered to 7.0 or lower, 6.5 or lower, 6.0 or lower, 5.5 or lower, 5.0 or lower, 4.5 or lower, 4.0 or lower, 3.5 or lower, 3.0 or lower, 2.5 or lower, 2.0 or lower, 1.5 or lower, 1.0 or lower, or as low as 0.5. In some embodiments, the pH is increased to 8.0 or higher, 8.5 or higher, 9.0 or higher, 9.5 or higher, 10.0 or higher, 10.5 or higher, 11.0 or higher, 11.5 or higher, 12.0 or higher, 12.5 or higher, 13.0 or higher, 13.5 or higher, or up to 14.0. In some embodiments, the pH of the composition or portion is adjusted to restore it to a pH at or near the pH the composition had prior to the purity-reducing process, before the composition or portion is added to a column or deformulated to separate nucleic acids. In some embodiments, the pH of the composition is adjusted to a pH of 6.5-8.0, 6.7-7.6, 7.0-7.5, or 7.2-7.4.

In some embodiments, the purity-reducing process increases the amount of impurities (e.g., mRNA fragments) in a composition by about 20% to about 1,000%. In some embodiments, the purity-reducing process increases the amount of impurities by about 25% to about 900%, about 30% to about 800%, about 35% to about 700%, about 40% to about 600%, about 45% to about 500%, about 50% to about 400%, about 55% to about 300%, about 60% to about 200%, about 65% to about 150%, or about 70% to about 100%.

In some embodiments, the methods described by the disclosure have a quantitation limit for contaminating mRNA fragments and lipid adducts that is 2.0% or less than the amount of target mRNA in a composition. A quantitation limit, with respect to a contaminant (e.g., mRNA fragments) and a target analyte (e.g., target mRNA), refers to the lowest amount of the contaminant, relative to the target analyte, that can reliably be detected and quantified. In some embodiments, the method has a quantitation limit for lipid adducts that is 1.5% or less, 1.0% or less, 0.8% or less, 0.7% or less, 0.6% or less, 0.5% or less, 0.4% or less, 0.3% or less, 0.2% or less, or as low as 0.1% of the amount of target mRNA in the composition. In some embodiments, the method has a quantitation limit for mRNA fragments that is 1.8% or less, 1.7% or less, 1.6% or less, 1.5% or less, 1.4% or less, 1.3% or less, 1.2% or less, 1.1% or less, 1.0% or less, 0.8% or less, 0.6% or less, or as low as 0.5% of the amount of target mRNA in the composition.

A detection limit, with respect to a contaminant (e.g., mRNA fragments) and a target analyte (e.g., target mRNA), refers to the lowest amount of the contaminant, relative to the target analyte, that can reliably be detected. In some embodiments, the limit of detection of a contaminant is 20-50%, 25-40%, or 30-35% the quantitation limit of the contaminant. In some embodiments, the method has a quantitation limit for lipid adducts that is 0.5% or less, 0.4% or less, 0.3% or less, 0.2% or less, 0.1% or less, 0.05% or less, or as low as 0.01% of the amount of target mRNA in the composition. In some embodiments, the method has a quantitation limit for mRNA fragments that is 0.6% or less, 0.5% or less, 0.4% or less, 0.3% or less, 0.2% or less, or as low as 0.1% of the amount of target mRNA in the composition.

In some embodiments, a method described by the disclosure is capable of accurately measuring target mRNA concentrations in a composition from a range of 0.002 mg/mL to 0.300 mg/mL. In some embodiments, a method may be evaluated by performing serial dilutions of a composition, analyzing each dilution using a method described herein, and performing linear regression of the relationship between the calculated target mRNA concentration in the composition and the % area under the curve corresponding to the target mRNA (relative to the % area under the curve of the undiluted composition). A coefficient of determination (R²) of 0.99 or higher in a linear regression between calculated target mRNA concentration and relative % area under the target mRNA chromatogram curve indicates that the method is capable of accurately measuring target mRNA concentrations in compositions having between the lowest calculated mRNA concentration in the dilution series and the target mRNA concentration in the undiluted composition. In some embodiments, the method is capable of accurately measuring target mRNA in compositions comprising 0.002 mg/mL to 0.300 mg/mL, 0.005 mg/mL to 0.250 mg/mL, 0.010 mg/mL to 0.200 mg/mL, 0.020 mg/mL to 0.150 mg/mL, or 0.05 mg/mL to 0.100 mg/mL.

In some embodiments, the nucleic acids of are formulated as a lipid composition, such as a composition comprising a lipid nanoparticle, a liposome, and/or a lipoplex. In some embodiments, nucleic acids of the invention are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

In some embodiments, the ionizable amino lipid of the present disclosure is a compound of Formula (AI):

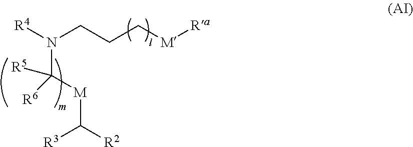

or its N-oxide, or a salt or isomer thereof,
wherein R′^{a} is R′^{branched}; wherein
R′^{branched} is:

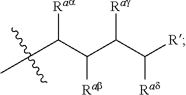

wherein

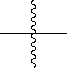

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

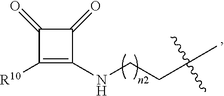

wherein

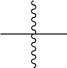

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

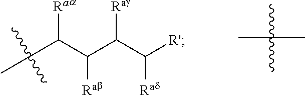

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

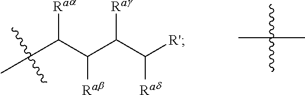

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

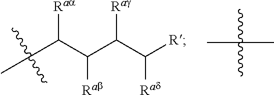

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

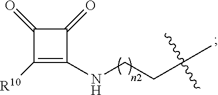

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}; R′^{branched} is

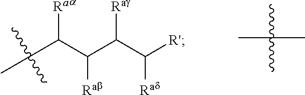

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

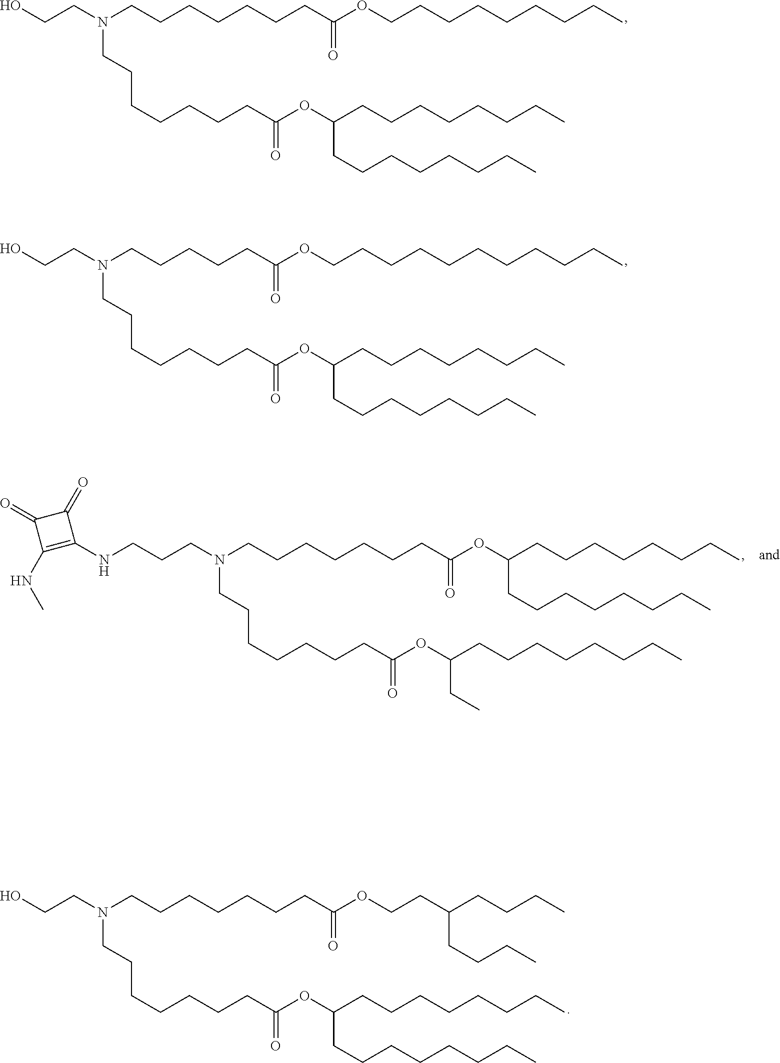 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid is a compound of Formula (AIa):

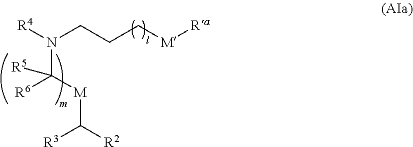

or its N-oxide, or a salt or isomer thereof, wherein R′^{a} is R′^{branched}; wherein
R′^{branched} is:

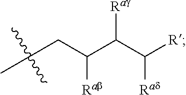

wherein

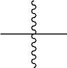

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and

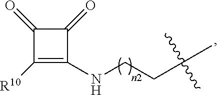

wherein

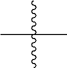

denotes a point of attachment; wherein
   R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.
In some embodiments, the ionizable amino lipid is a compound of Formula (AIb):

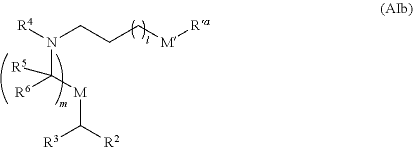

or its N-oxide, or a salt or isomer thereof,
wherein R′^{a} is R′^{branched}; wherein
R′^{branched} is:

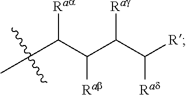

wherein

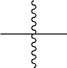

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

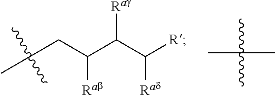

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

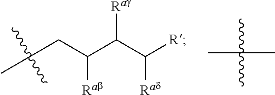

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (Ab), R′^{a} is R′^{branched}; R′^{branched} is

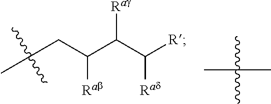

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid is a compound of Formula (AIc):

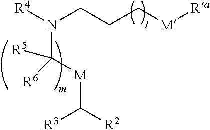

or its N-oxide, or a salt or isomer thereof,
wherein R′^{a} is R′^{branched}; wherein
   R′^{branched} is:

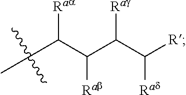

wherein

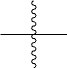

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

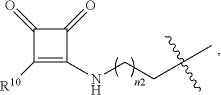

wherein

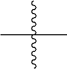

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

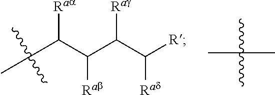

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

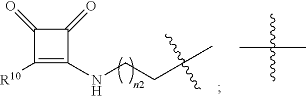

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

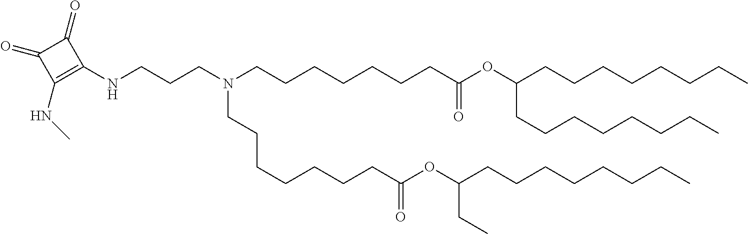 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

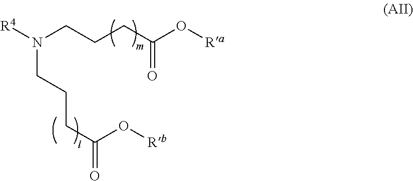

or its N-oxide, or a salt or isomer thereof,
wherein R′^{a} is R′^{branched} or R′^{cyclic}; wherein
R′^{branched} is:

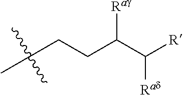

and R′^{cyclic} is:

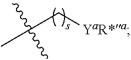

and

R′^{b} is:

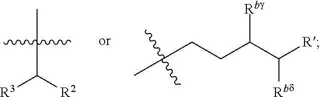

wherein

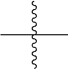

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

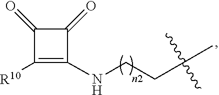

wherein

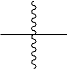

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-a):

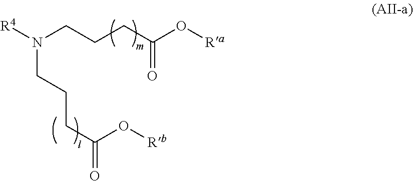

or its N-oxide, or a salt or isomer thereof,
wherein R′^{a} is R′^{branched} or R′^{cyclic}; wherein
R′^{branched} is:

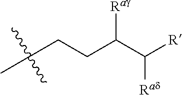

and R′^{b} is:

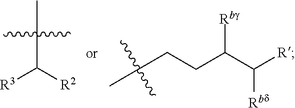

wherein

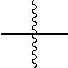

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

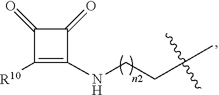

wherein

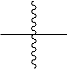

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-b):

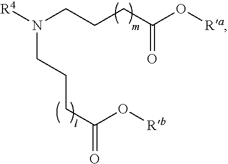

or its N-oxide, or a salt or isomer thereof,
wherein R′^{a} is R′^{branched} or R′^{cyclic}; wherein
R′^{branched} is:

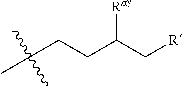

and R′^{b} is:

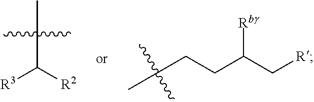

wherein

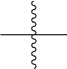

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

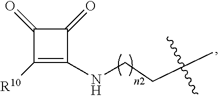

wherein

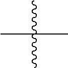

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-c):

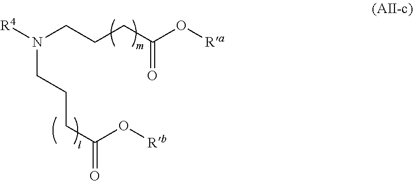

or its N-oxide, or a salt or isomer thereof,
wherein R′^{a} is R′^{branched} or R′^{cyclic}; wherein
R′^{branched} is:

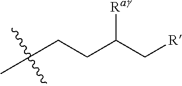

and R′^{b} is:

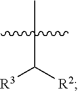

wherein

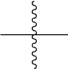

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and

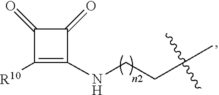

wherein

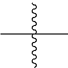

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-d):

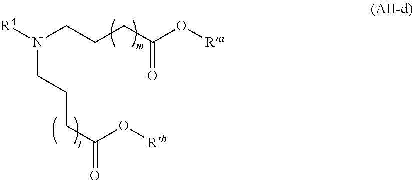

or its N-oxide, or a salt or isomer thereof,
wherein R′^{a} is R′^{branched} or R′^{cyclic}; wherein
R′^{branched} is:

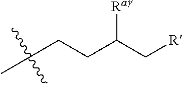

and R′^{b} is:

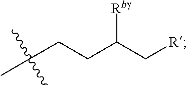

wherein

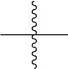

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and

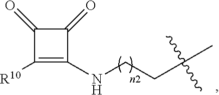

wherein

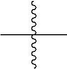

denotes a point of attachment; wherein R¹⁰ is N(R)₂; each R is independently selected prom t e group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-e):

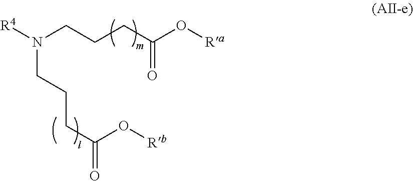

or its N-oxide, or a salt or isomer thereof,
wherein R′^{a} is R′^{branched} or R′^{cyclic}; wherein
R′^{branched} is:

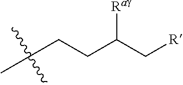

and R′^{b} is:

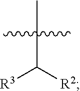

wherein

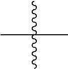

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

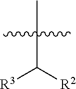

and R² and R³ are each independently a C₁₋₁₄ alkyl. In some embodiments of the compound of Formula (AII-a), (AII-b), (AII-c), (AII-d), or (AII-e), R′^{b} is:

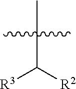

and R² and R³ are each independently a C₆₋₁₀ alkyl. In some embodiments of the compound of Formula (AII), (AII-a), (AII-b), (AII-c), (AII-d), or (AII-e), R′^{b} is:

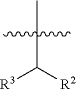

and R² and R³ are each a C₈ alkyl.

In some embodiments of the compound of Formula (AII), (AII-a), (AII-b), (AII-c), (AII-d), or (AII-e), R′^{branched} is:

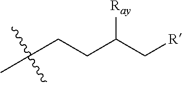

### and R′^{b} is:

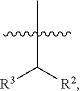

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

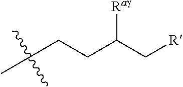

### and R′^{b} is:

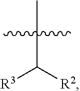

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

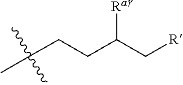

### and R′^{b} is:

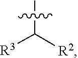

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

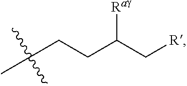

### R′^{b} is:

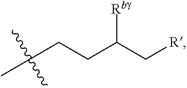

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

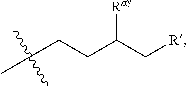

### R′^{b} is:

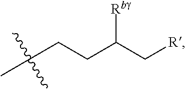

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

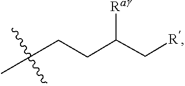

### R′^{b} is:

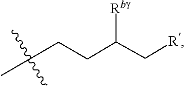

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

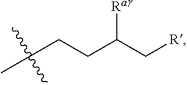

### R′^{b} is:

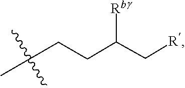

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

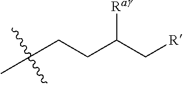

### and R′^{b} is:

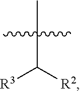

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

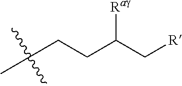

### and R′^{b} is:

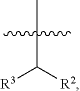

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

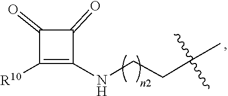

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

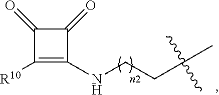

wherein R¹⁰ is NH(CH₃) and n2 is 2.

In some embodiments of the compound of Formula (AII), (AII-a), (AII-b), (AII-c), (AII-d), or (AII-e), R′^{branched} is:

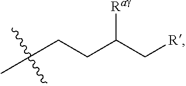

### R′^{b} is:

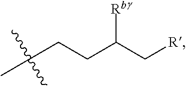

m and l are each independently selected from 4, 5, and 6, each R′ independently is a C₁₋₁₂ alkyl, R^{aγ} and R^{bγ} are each a C₁₋₁₂ alkyl, and R⁴ is

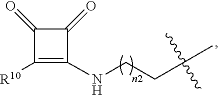

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

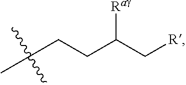

### R′^{b} is:

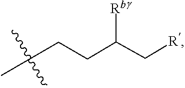

m and l are each 5, each R′ independently is a C₂₋₅ alkyl, R^{aγ} and R^{bγ} are each a C₂₋₆ alkyl, and R⁴ is

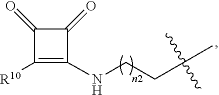

wherein R¹⁰ is NH(CH₃) and n2 is 2.

In some embodiments of the compound of Formula (AII), (AII-a), (AII-b), (AII-c), (AII-d), or (AII-e), R′^{branched} is:

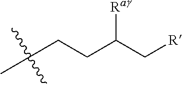

### and R′^{b} is:

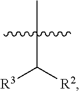

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

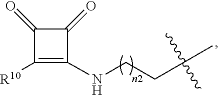

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

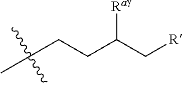

### and R′^{b} is:

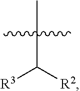

m and l are each 5, R′ is a C₂₋₅ alkyl, R^{aγ} is a C₂₋₆ alkyl, R² and R³ are each a C₈ alkyl, and R⁴ is

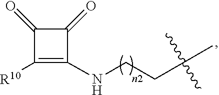

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

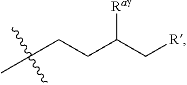

### R′^{b} is:

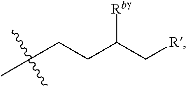

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

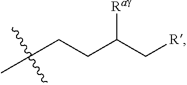

R′^{b} is:

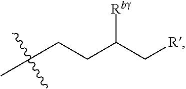

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid is a compound of Formula (AII-f):

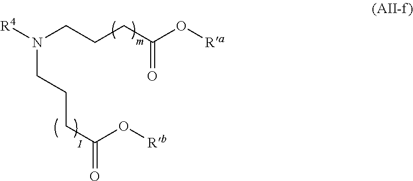

or its N-oxide, or a salt or isomer thereof,
wherein R′^{a} is R′^{branched} or R′^{cyclic}; wherein
R′^{branched} is:

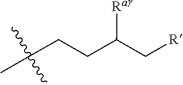

and R′^{b} is:

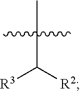

wherein

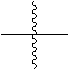

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid is a compound of Formula (AII-g):

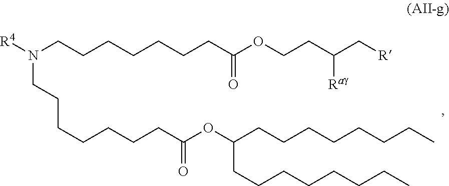

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

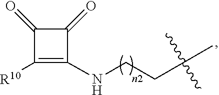

wherein

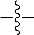

denotes a point of attachment, R¹⁰ is NH(C₁₋₆ alkyl), and n2 is selected from the group consisting of 1, 2, and 3.

In some embodiments, the ionizable amino lipid is a compound of Formula (AII-h):

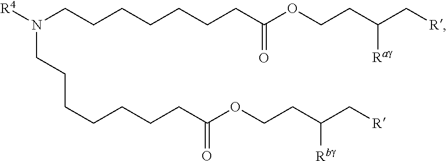

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

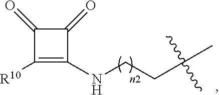

wherein

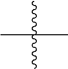

denotes a point of attachment, R₁₀ is NH(C₁₋₆ alkyl), and n2 is selected from the group consisting of 1, 2, and 3.

In some embodiments of the compound of Formula (AII-g) or (AII-h), R⁴ is

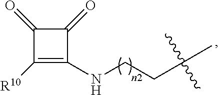

wherein
R¹⁰ is NH(CH₃) and n2 is 2.

In some embodiments of the compound of Formula (AII-g) or (AII-h), R⁴ is —(CH₂)₂OH.

In some embodiments, the ionizable amino lipids of the present disclosure may be one or more of compounds of Formula (VI):

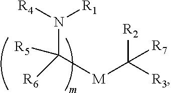

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)·CHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)·CHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (VI) includes those of Formula (VI-A):

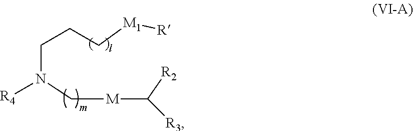

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VI-B):

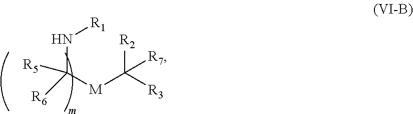

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VII):

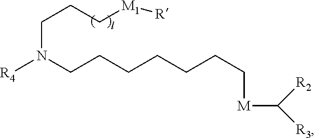

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (VI) are of Formula (VIIa),

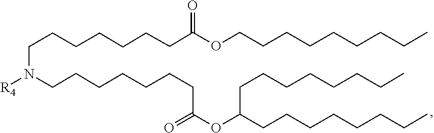

or their N-oxides, or salts or isomers thereof, wherein R₄ is as described herein.

In another embodiment, the compounds of Formula (VI) are of Formula (VIIb),

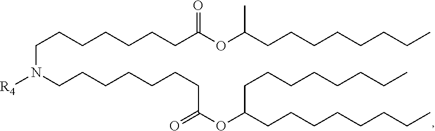

or their N-oxides, or salts or isomers thereof, wherein R₄ is as described herein.

In another embodiment, the compounds of Formula (VI) are of Formula (VIIc) or (VIIe):

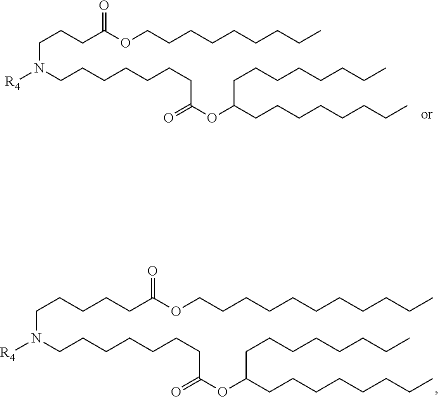

or their N-oxides, or salts or isomers thereof, wherein R₄ is as described herein.

In another embodiment, the compounds of Formula (VI) are of Formula (VIIf):

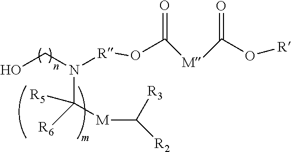

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (VI) are of Formula (VIId),

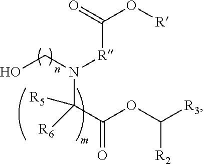

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

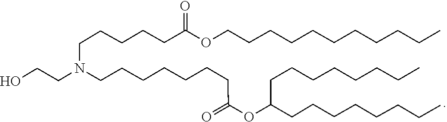 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

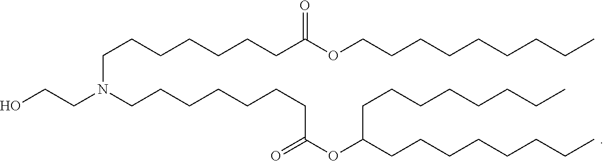 [SMILES: CCCCCCCCCOC(=O)CCCCCCCN(CCO)CCCCCCCC(=O)OC(CCCCCCCC)CCCCCCCC]

In a further embodiment, the compounds of Formula (VI) are of Formula (VIIg),

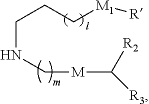

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (VI), (VI-A), (VI-B), (VII), (VIIa), (VIIb), (VIIc), (VIId), (VIIe), (VIIf), or (VIIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some embodiments, the ionizable amino lipids of the present disclosure may be one or more of compounds of formula (VIII),

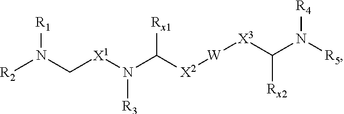

or salts or isomers thereof, wherein
W is

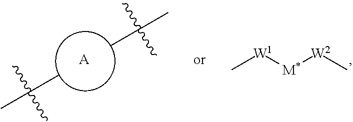

ring A is

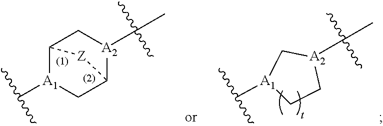

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃-6 carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

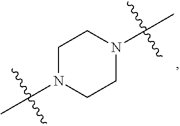 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (VIIIa1)-(VIIIa8):

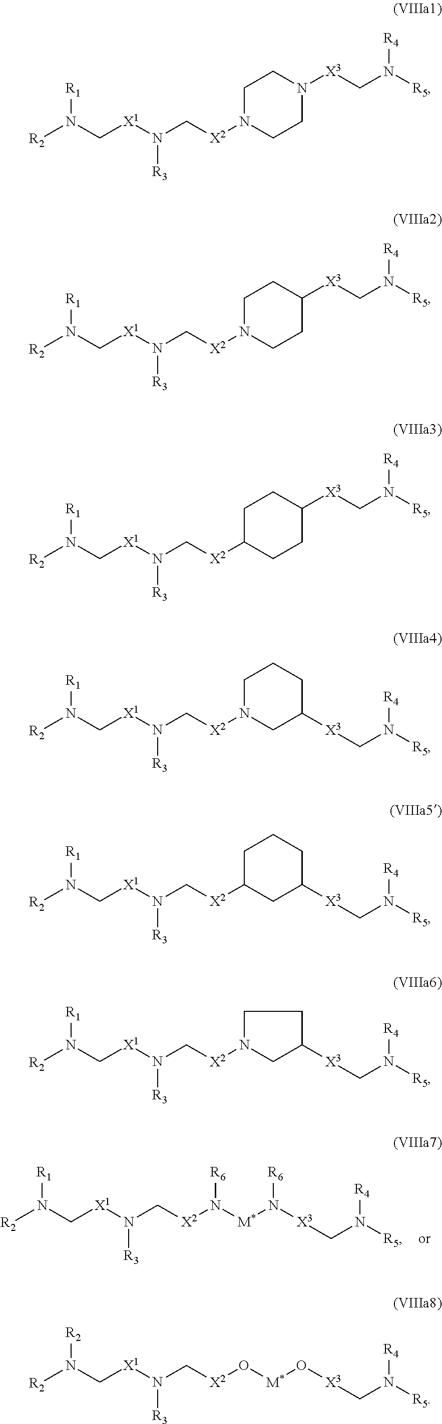

In some embodiments, the ionizable amino lipid is

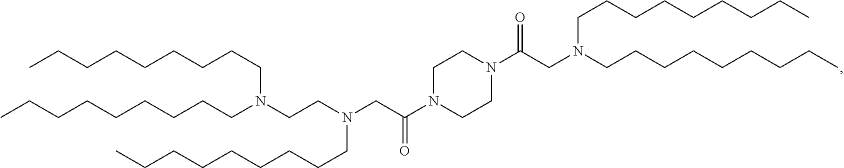 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (VIII), (VIIIa1), (VIIIa2), (VIIIa3), (VIIIa4), (VIIIa5), (VIIIa6), (VIIIa7), or (VIIIa8) may be protonated at a physiological pH. Thus, a lipid may have a positive or partial positive charge at physiological pH.

In some embodiments, the lipid nanoparticle comprises a lipid having the structure:

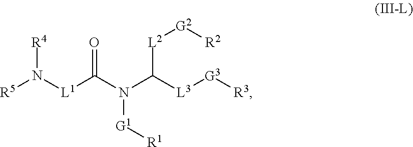

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or -0(C═O)—; and n is an integer greater than 0.

In some embodiments, the lipid nanoparticle comprises a lipid having the structure:

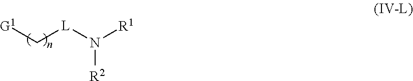

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

In some embodiments, the lipid nanoparticle comprises a lipid having the structure:

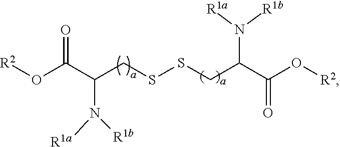

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CF₂)_{b}OCH₃;
each R³ and R⁴ is independently C₆-C₃₀ aliphatic;
each L₁ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

In some embodiments, the lipid nanoparticle comprises a lipid having the structure:

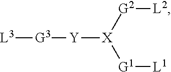

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}f; —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

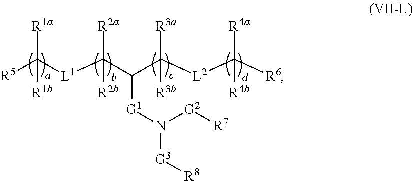

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-ₛ —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is H or C,-C₂₀ alkyl;
R⁸ is OH, —N(R⁹)(C=0)R¹⁰, —(C=0)NR⁹R¹⁰, —NR⁹R¹⁰, —(C=0)0R″¹ or -0(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

In some embodiments, the lipid nanoparticle comprises a lipid having the structure:

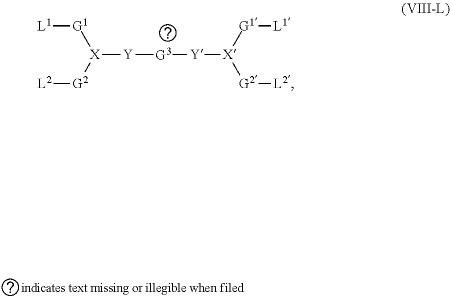

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
   a) Y is absent when X is N;
   b) Y′ is absent when X′ is N;
   c) Y is —O(C═O)—, —(C═O)O— or NR when X is CR; and
   d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R′ or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹. G^{1′}, G² and G^{2′} are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

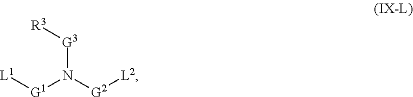

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R².
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

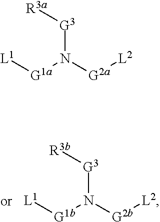

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

In some embodiments, the lipid nanoparticle comprises a lipid having the structure:

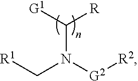

or a pharmaceutically acceptable salt, prodrug or stereoisomer thereof, wherein:
G¹ is —OH, —R³R⁴, —(C=0) R⁵ or —R³(C=0)R⁵;
G² is —CH₂— or —(C=0)-;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated Ci-C₆ alkyl;
R⁵ is optionally substituted straight or branched, saturated or unsaturated Ci-C₆ alkyl; and
n is an integer from 2 to 6.

In some embodiments, the lipid nanoparticle comprises a lipid having the structure:

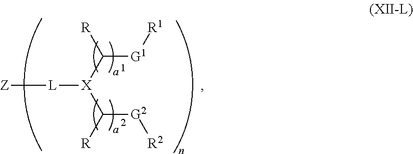

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ~O(C═O)—, wherein ~ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

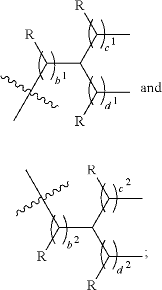

a¹ and a² are, at each occurrence, independently an integer from 3 to 12; b¹ and b² are a each occurrence, independently 0 or 1; c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, the lipid nanoparticle comprises a lipid having the structure:

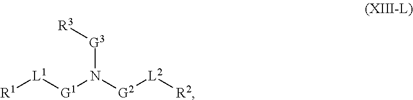

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O) R^{a}—, R^{a}C(═O) R^{a}—, —OC(═O) R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

In some embodiments, the lipid nanoparticle comprises a lipid having the structure:

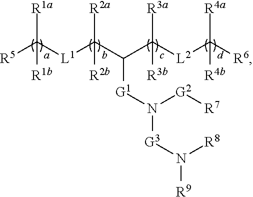

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In some embodiments, the lipid nanoparticle comprises a lipid having the structure:

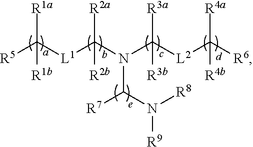

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In some embodiments, the lipid nanoparticle comprises a lipid having the structure:

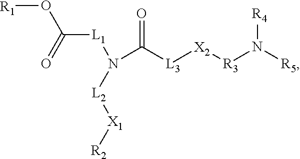

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

In some embodiments, the lipid nanoparticle comprises an ionizable lipid having the structure:

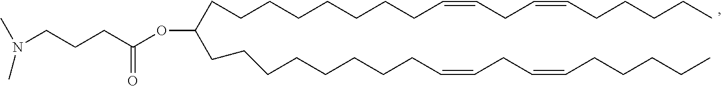 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

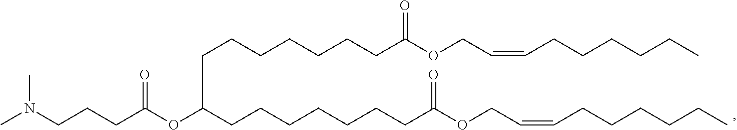 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt thereof.

In some embodiments, the lipid nanoparticle comprises a lipid having the structure:

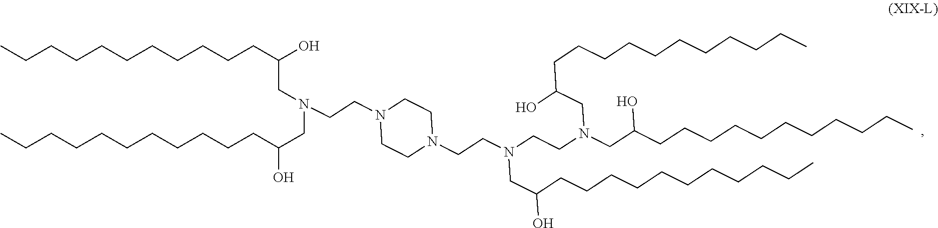 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

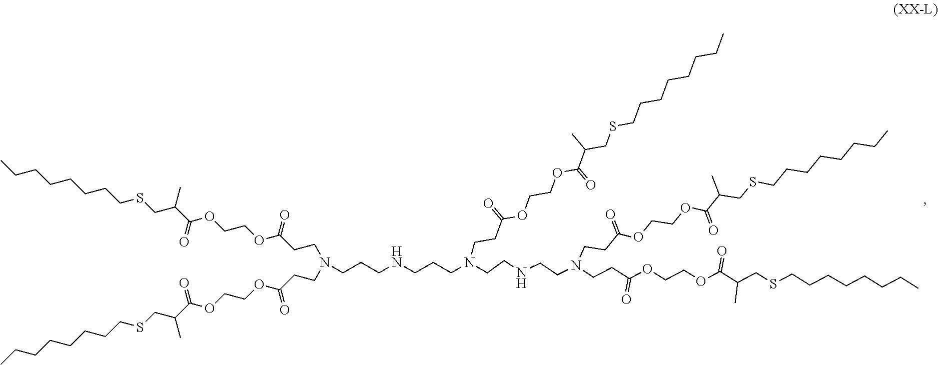 [SMILES: CCCCCCCCSCC(C)C(=O)OCCOC(=O)CCN(CCCNCCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

or a pharmaceutically acceptable salt thereof.

In some embodiments, the lipid nanoparticle comprises a lipid having the structure:

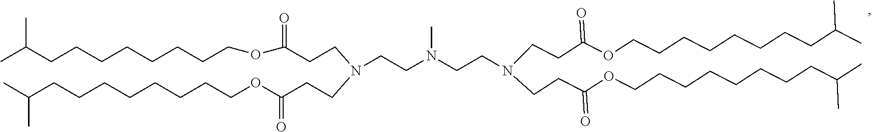 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

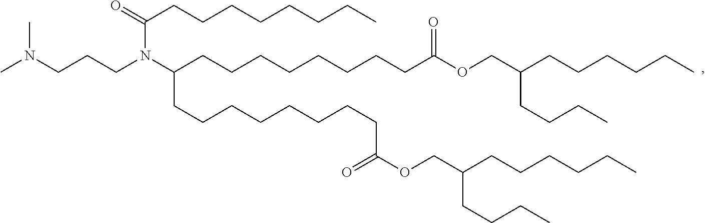 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

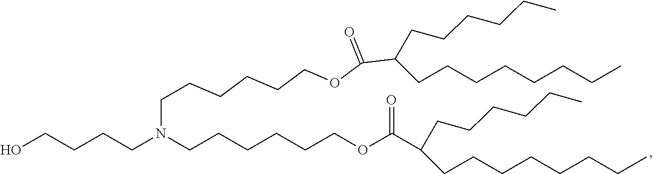 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

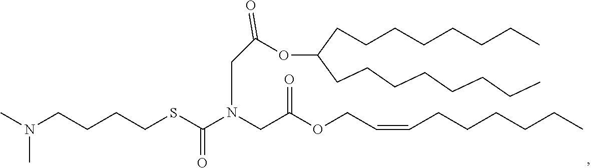 [SMILES: CCCCCC/C=C\COC(=O)CN(CC(=O)OC(CCCCCCCC)CCCCCCCC)C(=O)SCCCCN(C)C]

or a pharmaceutically acceptable salt thereof.

In some embodiments, the lipid nanoparticle comprises a lipid having the structure:

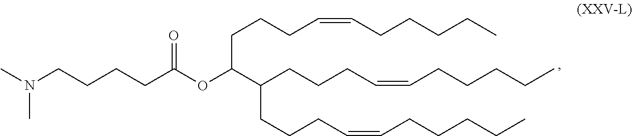 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

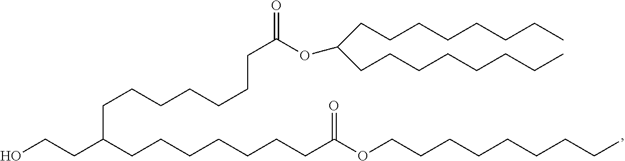

or a pharmaceutically acceptable salt thereof.

In some embodiments the lipid nanoparticle comprises a lipid having the structure:

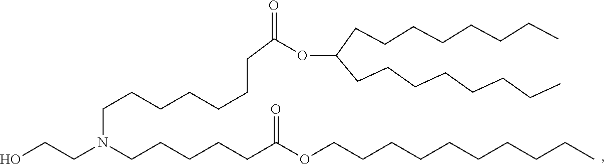 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In certain embodiments, a phospholipid useful or potentially useful in the present invention is an analog or variant of DSPC. In certain embodiments, a phospholipid useful or potentially useful in the present invention is a compound of Formula (IX):

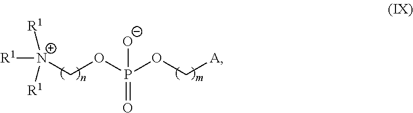

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

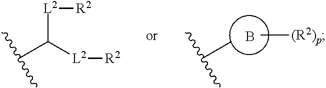

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁-30 alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

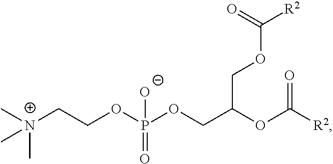

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 34-35 mol %, 35-36 mol %, 36-37 mol %, 37-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 Å2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

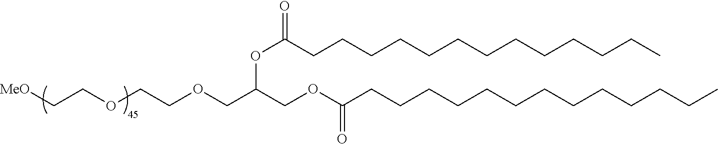

In some embodiments, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (X):

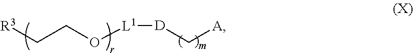

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

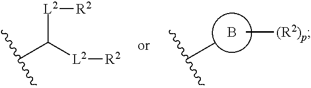

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (X) is a PEG-OH lipid (i.e., R³ is —OR^{O}, and R^{O} is hydrogen). In certain embodiments, the compound of Formula (X) is of Formula (X—OH):

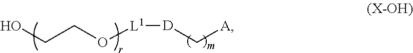

or a salt thereof.

In certain embodiments, a PEG lipid useful in the present invention is a PEGylated fatty acid. In certain embodiments, a PEG lipid useful in the present invention is a compound of Formula (XI). Provided herein are compounds of Formula (XI):

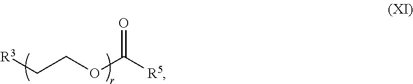

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (XI) is of Formula (XI-O

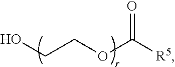

or a salt thereof. In some embodiments, r is 40-50.

In yet other embodiments the compound of Formula (XI) is:

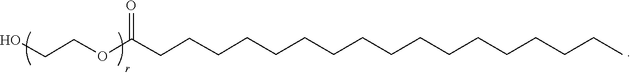

or a salt thereof.

In some embodiments, the compound of Formula (XI) is

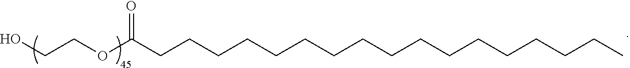

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). Without being bound by theory, it is believed that spiking a LNP composition with additional PEG can provide benefits during lyophilization. Thus, some embodiments, comprise adding additional PEG as compared to an amount used for a non-lyophilized LNP composition. In embodiments comprise adding about 0.5 mo % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP of the disclosure comprises an ionizable amino lipid of Compound 1, wherein the non-cationic lipid is DSPC, the structural lipid that is cholesterol, and the PEG lipid is DMG-PEG.

In some embodiments, a LNP of the invention comprises an ionizable amino lipid of any of Formula VI, VII or VIIII, a phospholipid comprising DSPC, a structural lipid, and a PEG lipid comprising PEG-DMG.

In some embodiments, a LNP of the invention comprises an ionizable amino lipid of any of Formula VI, VII or VIII, a phospholipid comprising DSPC, a structural lipid, and a PEG lipid comprising a compound having Formula XI.

In some embodiments, a LNP of the invention comprises an ionizable amino lipid of Formula VI, VII or VIII, a phospholipid comprising a compound having Formula VIII, a structural lipid, and the PEG lipid comprising a compound having Formula X or XI.

In some embodiments, a LNP of the invention comprises an ionizable amino lipid of Formula VI, VII or VIII, a phospholipid comprising a compound having Formula IX, a structural lipid, and the PEG lipid comprising a compound having Formula X or XI.

In some embodiments, a LNP of the invention comprises an ionizable amino lipid of Formula VI, VII or VIII, a phospholipid having Formula IX, a structural lipid, and a PEG lipid comprising a compound having Formula XI.

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the ionizable amino lipid component to the RNA of from about 10:1 to about 100:1.

In some embodiments, a LNP of the invention comprises a wt/wt ratio of the ionizable amino lipid component to the RNA of about 20:1.

In some embodiments, a LNP of the invention comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

In some embodiments, a subject to which a composition comprising a nucleic acid and a lipid, is administered is a subject that suffers from or is at risk of suffering from a disease, disorder or condition, including a communicable or non-communicable disease, disorder or condition. As used herein, “treating” a subject can include either therapeutic use or prophylactic use relating to a disease, disorder or condition, and may be used to describe uses for the alleviation of symptoms of a disease, disorder or condition, uses for vaccination against a disease, disorder or condition, and uses for decreasing the contagiousness of a disease, disorder or condition, among other uses.

In some embodiments the nucleic acid is an mRNA vaccine designed to achieve particular biologic effects. Exemplary vaccines of the invention feature mRNAs encoding a particular antigen of interest (or an mRNA or mRNAs encoding antigens of interest). In exemplary aspects, the vaccines of the invention feature an mRNA or mRNAs encoding antigen(s) derived from infectious diseases or cancers.

Diseases or conditions, in some embodiments include those caused by or associated with infectious agents, such as bacteria, viruses, fungi and parasites. Non-limiting examples of such infectious agents include Gram-negative bacteria, Gram-positive bacteria, RNA viruses (including (+)ssRNA viruses, (−)ssRNA viruses, dsRNA viruses), DNA viruses (including dsDNA viruses and ssDNA viruses), reverse transcriptase viruses (including ssRNA-RT viruses and dsDNA-RT viruses), protozoa, helminths, and ectoparasites.

Thus, the invention also encompasses infectious disease vaccines. The antigen of the infectious disease vaccine is a viral or bacterial antigen.

In some embodiments, a disease, disorder, or condition is caused by or associated with a virus.

The compositions of the invention are also useful for treating or preventing a symptom of diseases characterized by missing or aberrant protein activity, by replacing the missing protein activity or overcoming the aberrant protein activity. Because of the rapid initiation of protein production following introduction of mRNAs, as compared to viral DNA vectors, the compounds of the present disclosure are particularly advantageous in treating acute diseases such as sepsis, stroke, and myocardial infarction. Moreover, the lack of transcriptional regulation of the alternative mRNAs of the present disclosure is advantageous in that accurate titration of protein production is achievable. Multiple diseases are characterized by missing (or substantially diminished such that proper protein function does not occur) protein activity. Such proteins may not be present, are present in very low quantities or are essentially non-functional. The present disclosure provides a method for treating such conditions or diseases in a subject by introducing polynucleotide or cell-based therapeutics containing the alternative polynucleotides provided herein, wherein the alternative polynucleotides encode for a protein that replaces the protein activity missing from the target cells of the subject.

Diseases characterized by dysfunctional or aberrant protein activity include, but are not limited to, cancer and other proliferative diseases, genetic diseases (e.g., cystic fibrosis), autoimmune diseases, diabetes, neurodegenerative diseases, cardiovascular diseases, and metabolic diseases. The present disclosure provides a method for treating such conditions or diseases in a subject by introducing polynucleotide or cell-based therapeutics containing the polynucleotides provided herein, wherein the polynucleotides encode for a protein that antagonizes or otherwise overcomes the aberrant protein activity present in the cell of the subject.

In some embodiments, a composition disclosed herein does not comprise a pharmaceutical preservative. In other embodiments, a composition disclosed herein does comprise a pharmaceutical preservative. Non-limiting examples of pharmaceutical preservatives include methyl paragen, ethyl paraben, propyl paraben, butyl paraben, benzyl alcohol, chlorobutanol, phenol, meta cresol (m-cresol), chloro cresol, benzoic acid, sorbic acid, thiomersal, phenylmercuric nitrate, bronopol, propylene glycol, benzylkonium chloride, and benzethionium chloride. In some embodiments, a composition disclosed herein does not comprise phenol, m-cresol, or benzyl alcohol. Compositions in which microbial growth is inhibited may be useful in the preparation of injectable formulations, including those intended for dispensing from multi-dose vials. Multi-dose vials refer to containers of pharmaceutical compositions from which multiple doses can be taken repeatedly from the same container. Compositions intended for dispensing from multi-dose vials typically must meet USP requirements for antimicrobial effectiveness.

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

To “treat” a disease as the term is used herein, means to reduce the frequency or severity of at least one sign or symptom of a disease, disorder or condition experienced by a subject. The compositions described above or elsewhere herein are typically administered to a subject in an effective amount, that is, an amount capable of producing a desirable result. The desirable result will depend upon the active agent being administered. For example, an effective amount of a composition comprising a nucleic acid and a lipid may be an amount of the composition that is capable of increasing expression of a protein in the subject. A therapeutically acceptable amount may be an amount that is capable of treating a disease or condition, e.g., a disease or condition that that can be relieved by increasing expression of a protein in a subject. As is well known in the medical and veterinary arts, dosage for any one subject depends on many factors, including the subject's size, body surface area, age, the particular composition to be administered, the active ingredient(s) in the composition, the intended outcome of the administration, time and route of administration, general health, and other drugs being administered concurrently.

In some embodiments, a subject is administered a composition comprising a nucleic acid and a lipid I in an amount sufficient to increase expression of a protein in the subject.

In certain embodiments, LNP preparations (e.g., populations or formulations) are analyzed for polydispersity in size (e.g., particle diameter) and/or composition (e.g., amino lipid amount or concentration, phospholipid amount or concentration, structural lipid amount or concentration, PEG-lipid amount or concentration, mRNA amount (e.g., mass) or concentration) and, optionally, further assayed for in vitro and/or in vivo activity. Fractions or pools thereof can also be analyzed for accessible mRNA and/or purity (e.g., purity as determined by reverse-phase (RP) chromatography).

Particle size (e.g., particle diameter) can be determined by Dynamic Light Scattering (DLS). DLS measures a hydrodynamic diameter. Smaller particles diffuse more quickly, leading to faster fluctuations in the scattering intensity and shorter decay times for the autocorrelation function. Larger particles diffuse more slowly, leading to slower fluctuations in the scattering intensity and longer decay times in the autocorrelation function.

mRNA purity can be determined by reverse phase high-performance liquid chromatography (RP-HPLC) size based separation. This method can be used to assess mRNA integrity by a length-based gradient RP separation and UV detection of RNA at 260 nm. As used herein “main peak” or “main peak purity” refers to the RP-HPLC signal detected from mRNA that corresponds to the full size mRNA molecule loaded within a given LNP formulation. mRNA purity can also be assessed by fragmentation analysis. Fragmentation analysis (FA) is a method by which nucleic acid (e.g., mRNA) fragments can be analyzed by capillary electrophoresis. Fragmentation analysis involves sizing and quantifying nucleic acids (e.g., mRNA), for example by using an intercalating dye coupled with an LED light source. Such analysis may be completed, for example, with a Fragment Analyzer from Advanced Analytical Technologies, Inc.

Compositions formed via the methods described herein may be particularly useful for administering an agent to a subject in need thereof. In some embodiments, the compositions are used to deliver a pharmaceutically active agent. In some instances, the compositions are used to deliver a prophylactic agent. The compositions may be administered in any way known in the art of drug delivery, for example, orally, parenterally, intravenously, intramuscularly, subcutaneously, intradermally, transdermally, intrathecally, submucosally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions described herein and for use in accordance with the embodiments described herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, parenterally, intracisternally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also possible.

The ointments, pastes, creams, and gels may contain, in addition to the compositions of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compositions of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the compositions in a polymer matrix or gel.

In other embodiments, the compositions of the invention are loaded and stored in prefilled syringes and cartridges for patient-friendly autoinjector and infusion pump devices.

Kits for use in preparing or administering the compositions are also provided. A kit for forming compositions may include any solvents, solutions, buffer agents, acids, bases, salts, targeting agent, etc. needed in the composition formation process. Different kits may be available for different targeting agents. In certain embodiments, the kit includes materials or reagents for purifying, sizing, and/or characterizing the resulting compositions. The kit may also include instructions on how to use the materials in the kit. The one or more agents (e.g., pharmaceutically active agent) to be contained within the composition are typically provided by the user of the kit.

Kits are also provided for using or administering the compositions. The compositions may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the compositions. Exemplary devices include syringes, spoons, measuring devices, etc. The kit may optionally include instructions for administering the compositions (e.g., prescribing information).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As disclosed herein, the terms “composition” and “formulation” are used interchangeably.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., ±10% of the stated value, including 10% below and 10% above)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

### Example 1: HPLC Methods to Assay Active Ingredient by Injecting Drug Product

Studies exploring the feasibility of directly injecting drug products (e.g., a therapeutic and/or prophylactic nucleic acid formulated in a lipid nanoparticle pharmaceutical composition) in RP-HPLC experiments to assay the identity, stability, purity, etc. of the active ingredient (e.g., the therapeutic or prophylactic nucleic acid), without extracting the active ingredient, were performed.

TABLE 1
One embodiment of a method for direct injection of the drug
product to assay the active ingredient is described.

| | |
|---|---|
| Mobile Phase A | 250 mM tetrapropylammonium bromide, 250 mM |
| (MPA) | tetrabutylammonium chloride, pH 8 |
| Mobile Phase B | 250 mM tetrapropylammonium bromide, 250 mM |
| (MPB) | tetrabutylammonium chloride, 50% Acetonitrile, |
| | pH 8 |
| Column Temperature | 80 °C |
| Column | DNAPac RP 2.1 × 100 mm |
| Flow rate | 0.3 mL/min |

TABLE 2
A second embodiment of a method for direct injection of the
drug product to assay the active ingredient is described.

| | |
|---|---|
| Mobile Phase A | 250 mM tetrabutylammonium phosphate, 250 |
| (MPA) | mM triethylammonium acetate pH 5.6 |
| Mobile Phase B | 250 mM tetrabutylammonium phosphate, 250 |
| (MPB) | mM triethylammonium acetate pH 5.6 |
| Column Temperature | 65 °C |
| Column | Proswift RP-1S |
| Flow rate | 0.6 mL/min |

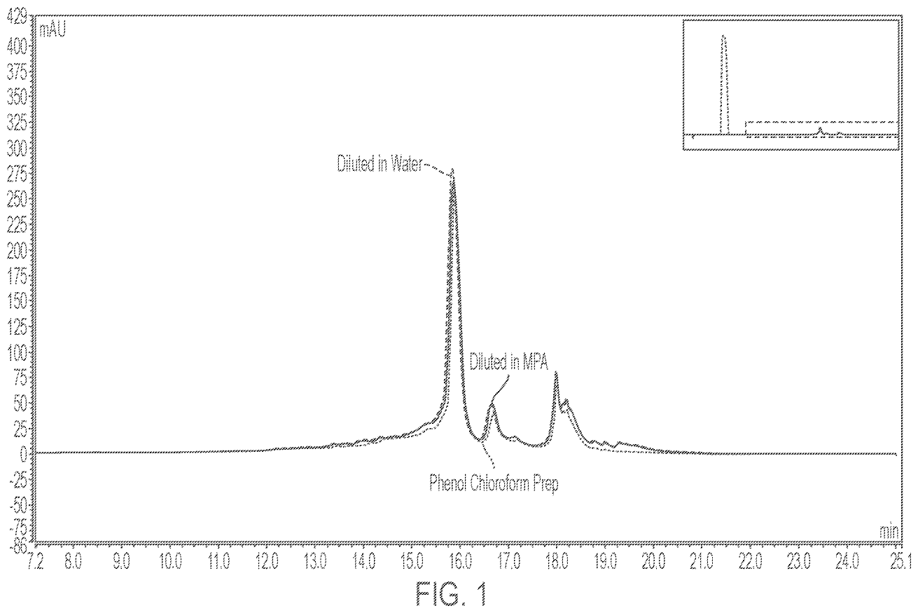

First, the method performance with and without mRNA extraction was tested. The adduct and main peak for the mRNA were as expected and the total peak area was within 10% of expected. The effect of diluting the lipid nanoparticle drug product in water or MPA on HPLC performance was compared with mRNA extracted from the lipid nanoparticle drug product using phenol-chloroform extraction. Similar peaks were observed in all three preparations (FIG. 1).

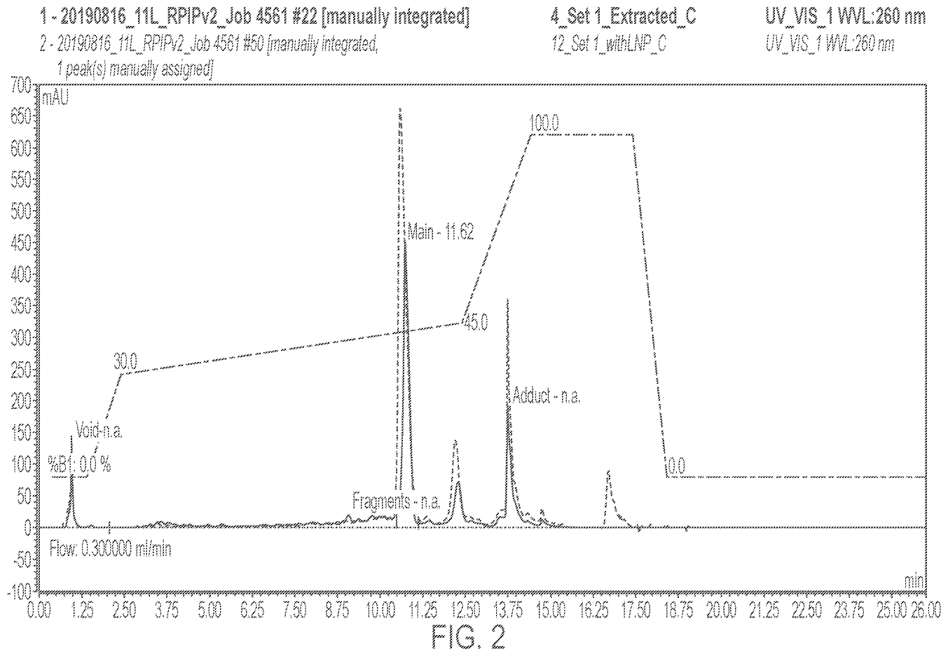

The ability of the system to resolve a complex mixture with direct injection was evaluated. A multivalent mRNA-LNP was analyzed with extraction by isopropanol precipitation and by direct injection. The same peak shape and retention time was observed for both preparations. Higher total signal with direct injection is indicative of loss in sample recovery through extraction (FIG. 2).

### Example 2: Effects of Mobile Phase Composition and Injection Volumes on HPLC Methods

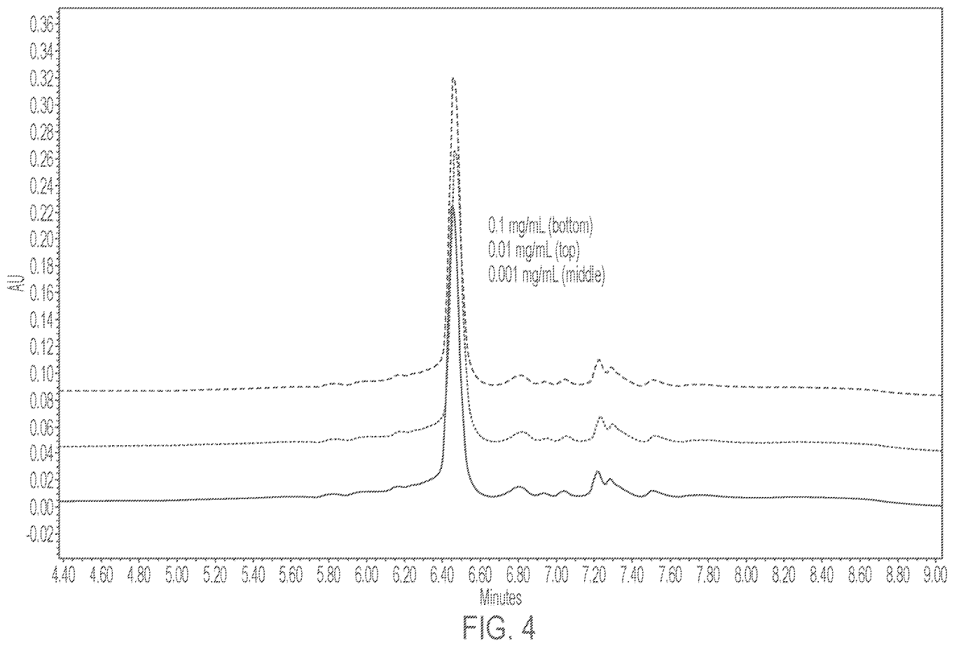

The ability of this method to analyze very dilute sample preparations such as low dose forms was assessed. Very low concentrations especially less than 0.1 mg/mL are challenging to recover through techniques such as precipitation or liquid:liquid extraction without additional sample manipulation. LNPs were diluted in formulation buffer to 0.1, 0.01, and 0.001 mg/mL and sample volumes of 10, 100, and 1000 uL were injected to achieve equal mass loading. Retention and peak shape was maintained showing robust performance of the method across sample concentrations (FIG. 4).

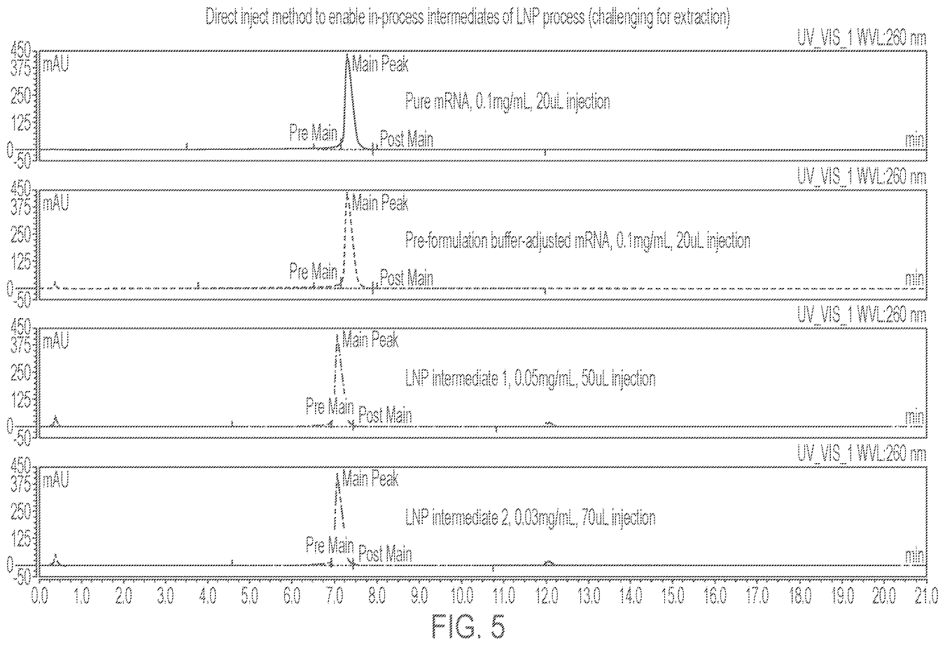

Method performance across LNP formulation process intermediates was assessed. LNP intermediates can provide additional challenges to sample extraction due to low concentrations and poor intermediate instability. The pure mRNA diluted to 0.1 mg/mL and pre-formulation buffer-adjusted mRNA at 0.1 mg/mL were analyzed alongside the first two LNP process intermediates at 0.05 mg/mL and 0.03 mg/mL. Injection volume was adjusted to target consistent column loading. Robust method performance was achieved for both early LNP process intermediates (FIG. 5).

### Example 3: Regeneration of HPLC Columns

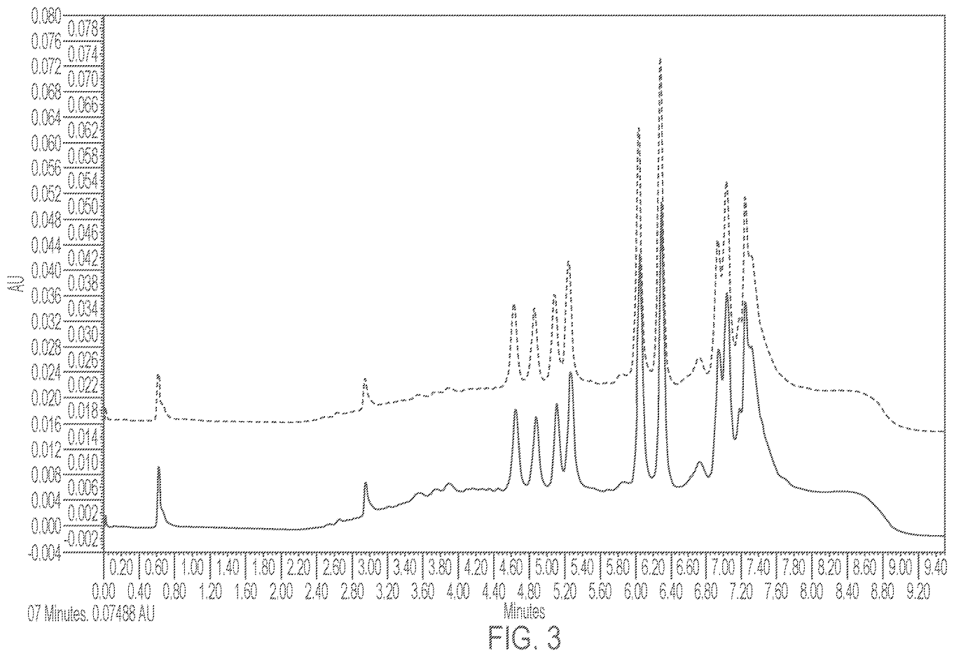

The ability of the column to withstand multiple injections was tested. Overlaid chromatograms are separated by over 200 direct inject LNP analyses, showing robust column performance over time without additional column washing required (FIG. 3).

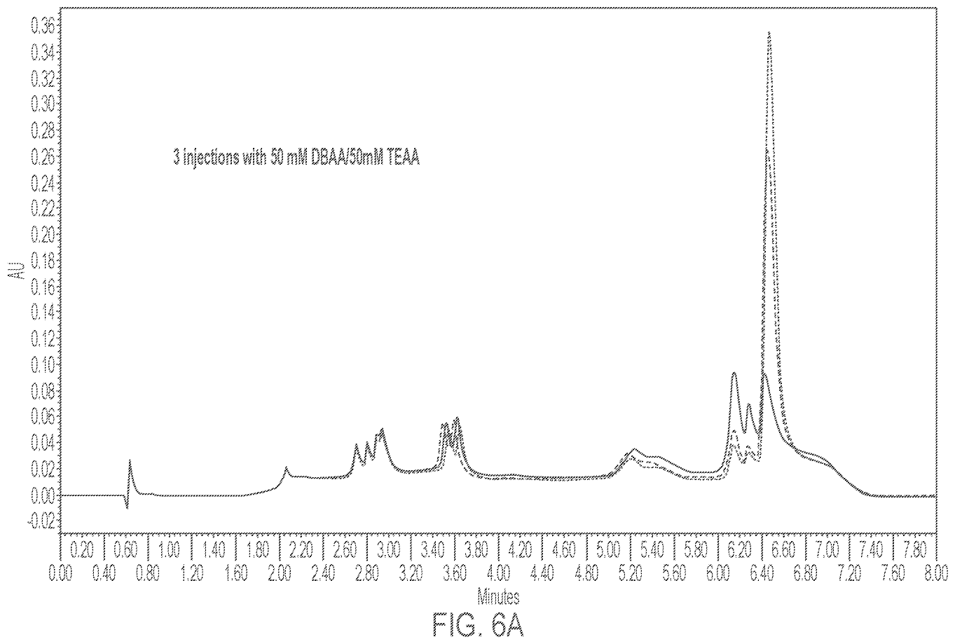

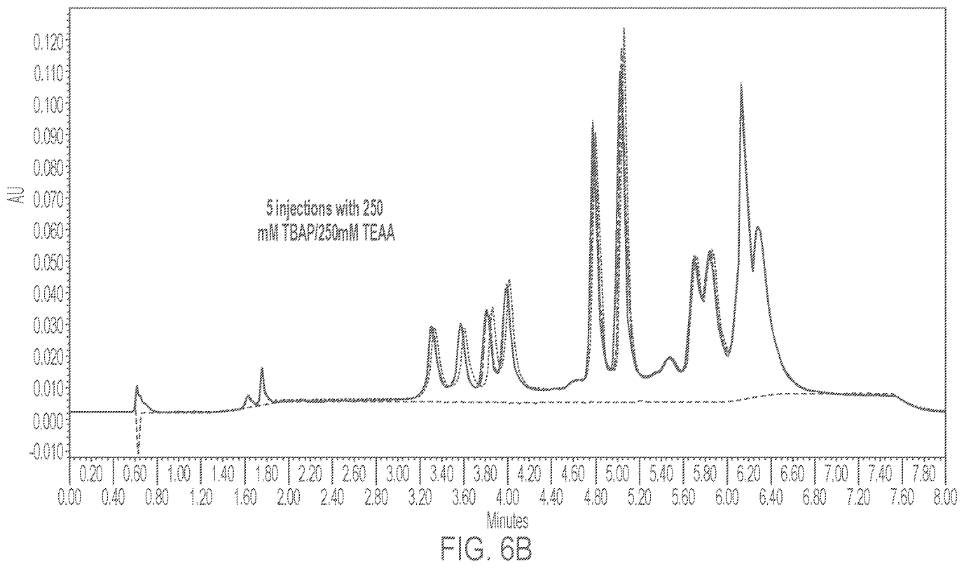

With inadequate mobile phase conditions, additional area in the high organic wash is one example of method failure. At 50 mM DBAA/50 mM TEAA, chromatographic area in the wash increases with three sequential injections indicating sample carryover, whereas at 250 mM TBAP/250 mM TEAA, consistent peak area is observed for five sequential injections (FIG. 6). These results indicate that a column can be used for multiple successive analyses, with consistent results, without the need for column regeneration between injections.

These experiments demonstrate that it is possible to perform RP-IP mRNA analysis on mRNA-LNPs to assay the mRNA integrity within the drug product without having to first extract the active ingredient using cumbersome extraction methods. This separation can surprisingly be performed in the presence of the lipid components, which strongly associate with the column due to their higher relative hydrophobicity. Mobile phase composition is important to optimize method selectivity and resolution, in addition to obtain robust method performance using the direct inject strategy.

### Example 4: Effects of Ion Pairing Agent Composition on HPLC Methods

The experiments described in Example 1 were adapted to determine the feasibility of using a single alkylammonium salt as an ion pairing agent for characterization of drug products by direct injection. In contrast to the methods tested in Example 1, mobile phases included only one alkylammonium salt as an ion pairing agent.

A range of concentrations of tetrabutylammonium bromide (TBAB) were tested, as shown in Table 3.

TABLE 3
A third embodiment of a method for direct injection of the
drug product to assay the active ingredient is described.

| | |
|---|---|
| Mobile Phase A | 50 mM-1M tetrabutylammonium bromide, pH 7 |
| (MPA) | |
| Mobile Phase B | 50 mM-1M tetrabutylammonium bromide (same |
| (MPB) | concentration as Mobile Phase A), 50% |
| | Acetonitrile, pH 7 |
| Column Temperature | 65 °C |
| Column | Proswift RP-1S |
| Flow rate | 0.75 mL/min |

Analytes tested included (1) mRNA in water; (2) mRNA formulated in a lipid nanoparticle (mRNA in LNP); (3) mRNA diluted in a composition containing 4 mg/mL lipid nanoparticles (mRNA with LNP); and (4) an mRNA that was separated from an LNP-mRNA composition using 60 mM ammonium acetate in IPA, then resuspended in water (“Extracted mRNA). This combination of samples was used to determine whether lipids present in a formulated sample interfered with the ability to resolve both mRNA and adduct purity.

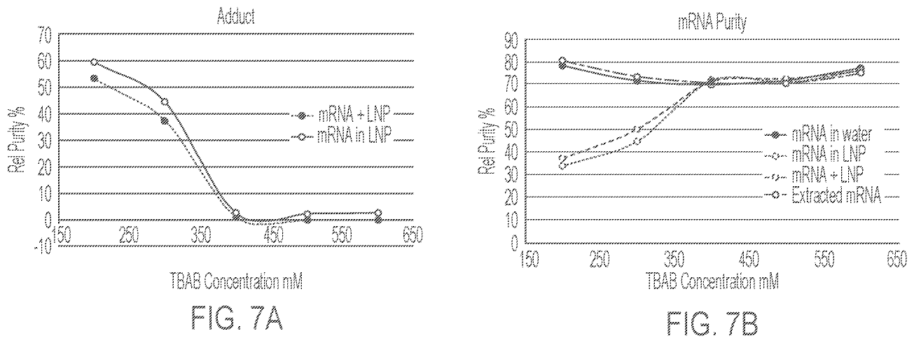

As concentration of the ion pairing agent increased, interference from lipids decreased, dropping to zero as the concentration exceeded 400 mM (FIG. 7A). Similarly, the observed purity of mRNA in LNP-mRNA compositions increased with the concentration of the ion pairing agent, becoming similar to the observed mRNA purity of lipid-free compositions once the ion pairing agent concentration reached 400 mM (FIG. 7B). Other alkylammonium salts, including tetramethylammonium chloride (TMAC), tetraethylammonium bromide (TEAB), tetrapropylammonium bromide (TPAB), tetrabutylammonium chloride (TBAC), tetrabutylammonium phosphate (TBAP), triethylammonium acetate (TEAA), dipropylammonium acetate (DPAA), dibutylammonium acetate (DBAA), and hexylammonium bromide (HAB), were similarly capable of separating lipid nanoparticle compositions and resolving lipid adducts. These results indicate that the capability of an ion pairing agent to separate a formulated sample (e.g., a nucleic acid formulated in a lipid nanoparticle) improves at increased concentrations.

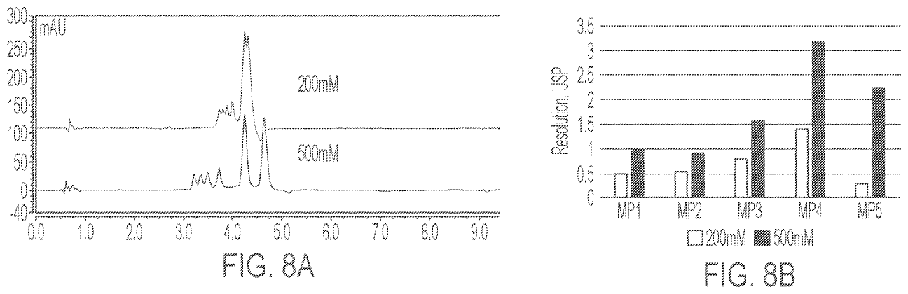

In a second experiment, the ability of methods to resolve five distinct mRNAs in a pentavalent mixture was evaluated. A lipid nanoparticle containing the mRNAs was deformulated using ammonium acetate in IPA, then resuspended in water, and reverse phase HPLC was used to quantify the mRNAs in the composition. When the concentration of alkylammonium salt in a composition increased from 200 mM to 500 mM, chromatography peaks separated to a greater extent, improving USP resolution between mRNAs (FIGS. 8A-8B). This improvement was most pronounced for longer mRNAs (see peaks 4 and 5 of FIG. 8A and columns 4 and 5 of FIG. 8B). These results indicate that higher concentrations of ion pairing agents improve the resolution of direct injection-based chromatography methods of analyzing formulate lipid nanoparticle drug products.

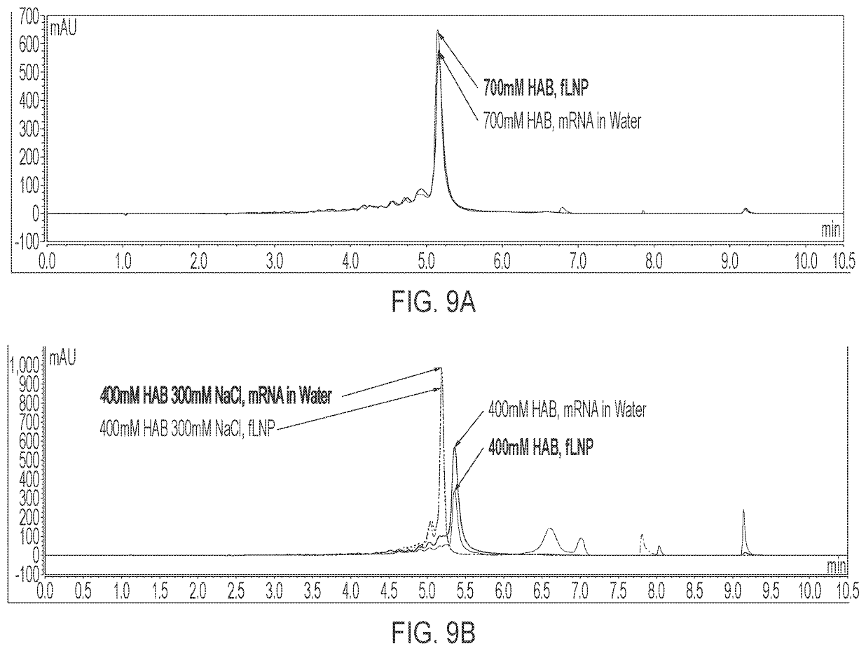

In a third experiment, an inorganic salt was added to the mobile phase to evaluate the contribution of salts to lipid nanoparticle deformulation. Analytes tested in this experiment included either (1) mRNA in water; or (2) mRNA formulated in a lipid nanoparticle (fLNP). In the absence of inorganic salts, 700 mM HAB was sufficient to deformulate LNPs, as both mRNA in water and fLNPs yielded similar chromatograms (FIG. 9A). At lower HAB concentrations, such as 400 mM, deformulation was incomplete, but the addition of 300 mM NaCl improved the extent of deformulation, as mRNA in water and fLNPs yielded similar chromatograms in mobile phases containing 400 mM HAB and 300 mM NaCl (FIG. 9B). These results indicate that inorganic salts (e.g., NaCl) also contribute to the deformulation of lipid nanoparticles, allowing the use of lower concentrations of alkylammonium salts required by direct injection RP-IP chromatography methods.

What is claimed is:

1. A method for identifying a target mRNA in a mixture, the method comprising:
(i) contacting a stationary phase of a reverse phase chromatography column with one or more mRNAs encapsulated in one or more lipid nanoparticles;
(ii) contacting the column with a mobile phase comprising a first solvent and a second solvent solution, each solvent solution comprising at least one ion pairing agent and at least one inorganic salt, wherein the second solvent solution comprises at least 50% v/v of an organic solvent, such that the target mRNA traverses the column with a retention time that is characteristic of the target mRNA;
(iii) detecting a signal corresponding to the retention time of the target mRNA; and
(iv) identifying the target mRNA as being present based upon detecting the signal corresponding to the retention time of the target mRNA,
wherein the method does not comprise extracting nucleic acids from the lipid nanoparticles prior to step (i).

2. The method of claim 1, wherein the at least one inorganic salt in the first and/or second solvent solutions is selected from the group consisting of a sodium salt, potassium salt, lithium salt, magnesium salt, calcium salt, and ammonium salt, optionally wherein the sodium salt is sodium chloride, sodium bromide, sodium acetate, sodium phosphate, or sodium sulfate, the potassium salt is potassium chloride, potassium bromide, potassium acetate, potassium phosphate, or potassium sulfate, the lithium salt is lithium chloride, lithium bromide, lithium acetate, lithium phosphate, or lithium sulfate, the magnesium salt is magnesium chloride, magnesium bromide, magnesium acetate, magnesium phosphate, or magnesium sulfate, the calcium salt is calcium chloride, calcium bromide, calcium acetate, calcium phosphate, or calcium sulfate, the ammonium salt is ammonium chloride, ammonium bromide, ammonium acetate, ammonium phosphate, or ammonium sulfate.

3. The method of claim 1 or 2, wherein the first and second solvent solutions comprise the same inorganic salt.

4. The method of any one of claims 1-3, wherein the concentration of each of the at least one inorganic salts in the first solvent solution and/or the second solvent solution ranges from about 0 mM-10M, 20 mM-9M, 30 mM-8M, 40 mM-7M, 50 mM-6M, 75 mM-5 M, 100 mM-2.5 M, 125 mM-2 M, 150 mM-1.5 M, 175 mM-1 M, or 200 mM-500 mM, optionally wherein the concentration of each of the at least one inorganic salts in the first solvent solution and/or the second solvent solution ranges from about 10 mM-1 M, 40 mM-300 mM, 50 mM-500 mM, 75 mM-400 mM, 100 mM-300 mM, 200-300 mM, 200-250 mM, or 250-300 mM.

5. The method of any one of claims 1-4, wherein each of the first and second solvent solutions comprises the same inorganic salt.

6. The method of any one of claims 1-5, wherein the first solvent solution and second solvent solution each comprise at least two ion pairing agents in a molar ratio of between about 1:10 to about 10:1, optionally wherein the first and/or second solvent solution are in a molar ratio between about 1:4 to about 4:1, about 1:5 to about 5:1, about 1:5 to about 5:1, about 1:3 to about 3:1, about 1:2 to about 2:1, or about 1:1.5 to about 1.5:1, optionally wherein the at least two ion pairing agents in the first and/or second solvent solution are in a 1:1 molar ratio.

7. The method of any one of claims 1-6, wherein the at least one ion pairing agent in the first and/or second solvent solution is selected from the group consisting of a trietheylammonium salt, tributylammonium salt, tetrabutylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt, optionally wherein the triethylammonium salt is triethylammonium acetate, the tetrabutylammonium salt is tetrabutylammonium phosphate, tetrabutylammonium phosphate, or tetrabutylammonium chloride, the hexylammonium salt is hexylammonium acetate or hexylammonium bromide, the dibutylammonium salt is dibutylammonium acetate, the tetrapropylammonium salt is dodecyltrimethylammonium chloride, the tetra(decyl)ammonium salt is tetra(decyl)ammonium bromide, the dihexylammonium salt is dihexylammonium acetate, the dipropylammonium salt is dipropylammonium acetate, the myristyltrimethylammonium salt is myristyltrimethylammonium bromide, the tetraethylammonium salt is tetraethylammonium bromide, the etraheptylammonium salt is tetraheptylammonium bromide, the tetrahexylammonium salt is tetrahexylammonium bromide, the tetrakis(decyl)ammonium salt is tetrakis(decyl)ammonium bromide, the tetramethylammonium salt is tetramethylammonium bromide, the tetraoctylammonium salt is tetraoctylammonium bromide, and/or the tetrapentylammonium salt is tetrapentylammonium bromide.

8. The method of claim 7, wherein the first solvent solution and the second solvent solution each comprise at least two ion pairing agents, wherein the at least two ion pairing agents are (i) tetrapropylammonium bromide and tetrabutylammonium chloride, (ii) dibutylammonium acetate and triethylammonium acetate, or (iii) tetrabutylammonium phosphate and triethylammonium acetate.

9. The method of any one of claims 1-8, wherein the concentration of each of the at least one ion pairing agents in the first solvent solution and/or the second solvent solution ranges from about 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM, optionally wherein the concentration of each of the at least one ion pairing agents in the first solvent solution and/or the second solvent solution ranges from about 10 mM-1M, 40 mM 300 mM, 50 mM-500 mM, 75 mM-400 mM, 100 mM-300 mM, 200-300 mM, 200-250 mM, or 250-300 mM.

10. The method of any one of claims 1-9, wherein the first solvent solution and/or the second solvent solution comprises 250 mM tetraproplyammonium bromide and 250 mM tetrabutylammonium chloride.

11. The method of any one of claims 1-5, wherein each of the first and second solvent solutions comprises a single alkylammonium salt and does not comprise more than one alkylammonium salt.

12. The method of claim 11, wherein the first and second solvent solutions comprise the same single alkylammonium salt.

13. The method of claim 11 or 12, wherein the single alkylammonium salt in the first and/or second solvent solutions is selected from the group consisting of a trietheylammonium salt, tributylammonium salt, tetrabutylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt, optionally wherein the triethylammonium salt is triethylammonium acetate, the tetrabutylammonium salt is tetrabutylammonium phosphate, tetrabutylammonium bromide, or tetrabutylammonium chloride, the hexylammonium salt is hexylammonium acetate or hexylammonium bromide, the dibutylammonium salt is dibutylammonium acetate, the tetrapropylammonium salt is dodecyltrimethylammonium chloride, the tetra(decyl)ammonium salt is tetra(decyl)ammonium bromide, the dihexylammonium salt is dihexylammonium acetate, the dipropylammonium salt is dipropylammonium acetate, the myristyltrimethylammonium salt is myristyltrimethylammonium bromide, the tetraethylammonium salt is tetraethylammonium bromide, the etraheptylammonium salt is tetraheptylammonium bromide, the tetrahexylammonium salt is tetrahexylammonium bromide, the tetrakis(decyl)ammonium salt is tetrakis(decyl)ammonium bromide, the tetramethylammonium salt is tetramethylammonium bromide, the tetraoctylammonium salt is tetraoctylammonium bromide, and/or the tetrapentylammonium salt is tetrapentylammonium bromide.

14. The method of any one of claims 11-13, wherein the first and second solvent solutions comprise a single alkylammonium salt selected from the group consisting of tetramethylammonium chloride, tetramethylammonium bromide, triethylammonium acetate, tetrapropylammonium bromide, dipropylammonium acetate, tributylammonium chloride, tetrabutylammonium chloride, tetrabutylammonium bromide, dibutylammonium acetate, and hexylammonium acetate.

15. The method of any one of claims 11-14, wherein the concentration of the single alkylammonium salt in each of the first and second solvent solutions ranges from about 50 mM-5 M, 100 mM-4 M, 200 mM-3 M, 300 mM-2 M, 400 mM-1M, 400 mM-800 mM, 400 mM-600 mM, or 400 mM-500 mM.

16. The method of any one of claims 11-15, wherein the single alkylammonium salt is selected from the group consisting of triethylammonium acetate, dipropylammonium acetate, tetrabutylammonium bromide, tetrabutylammonium phosphate, and hexylammonium bromide.

17. The method of any one of claims 11-16, wherein each of the first and second solvent solutions comprises:
(a) 400 mM-1.5 M triethylammonium acetate;
(b) 400 mM-1.5 M dipropylammonium acetate;
(c) 400 mM-1.5 M tetrabutylammonium bromide;
(d) 400 mM-1.5 M tetrabutylammonium phosphate; or
(e) 400 mM-1.5 M hexylammonium bromide.

18. The method of any one of claims 1-17, wherein the second solvent solution comprises about 50% to about 95%, about 55% to about 90%, about 60% to about 85%, about 65% to about 80%, or about 70% v/v to about 75% v/v of the organic solvent, optionally wherein the second solvent solution comprises about 50%, about 60%, about 70%, about 80%, or about 90% v/v of the organic solvent.

19. The method of any one of claims 1-18, wherein the organic solvent in the second solvent solution is selected from the group consisting of polar aprotic solvents, C₁₋₄ alkanols, C₁₋₆ alkanediols, and C₂₋₄ alkanoic acids.

20. The method of any one of claims 1-19, wherein the organic solvent in the second solvent solution is selected from the group consisting of acetonitrile, methanol, ethanol, isopropanol, acetone, propanol, tetrahydrofuran, dimethyl sulfoxide, dimethylformamide, and hexylene glycol, optionally wherein the organic solvent in the second solvent solution is acetonitrile.

21. The method of any one of claims 1-20, wherein the column is an analytical column, or a preparative column.

22. The method of any one of claims 1-21, wherein the stationary phase comprises particles, optionally wherein the particles are hydrophobic or comprise hydrophobic functional groups, optionally wherein the particles are porous resin particles.

23. The method of claim 22, wherein the particles have a diameter of about 2 μm-about 10 μm, about 2 μm-about 6 μm, or about 4 μm.

24. The method of claim 22 or 23, wherein the particles comprise pores having a diameter of about 500 Å to about 5000 Å, about 800 Å to about 3000 Å, or about 1000 Å to about 2000 Å.

25. The method of any one of claims 1-24, wherein the target mRNA is single-stranded.

26. The method of any one of claims 1-25, wherein the target mRNA comprises:
(i) 5′ and 3′ UTRs;
(ii) a 5′ cap, optionally wherein the 5′ cap is a 7-methylguanosine cap or a 7-methylguanosine group analog; and
(iii) a 3′ polyadenosine (polyA) tail.

27. The method of any one of claims 1-26, wherein the mRNA is in vitro transcribed (IVT) mRNA.

28. The method of any one of claims 1-27, wherein the target mRNA has a total length of between about 100 nucleotides and about 10,000 nucleotides, about 100 nucleotides to about 5,000 nucleotides, or about 200 nucleotides to about 4,000 nucleotides.

29. The method of any one of claims 1-28, wherein the pH of the first solvent solution and/or the second solvent solution is between about pH 6.8 and pH 9, optionally wherein the pH is about 8.0.

30. The method of any one of claims 1-29, wherein the column has a temperature from about 70 °C to about 90 °C, optionally wherein the column has a temperature of about 80 °C.

31. The method of any one of claims 1-30, wherein the volume percentage of the first solvent solution and volume percentage of the second solvent solution in the mobile phase are each varied from 0% to 100%.

32. The method of any one of claims 1-31, wherein the ratio of the first solvent solution to the second solvent solution is held constant during elution of the mRNA.

33. The method of any one of claims 1-32, wherein the ratio of the first solvent solution to the second solvent solution is increased or decreased during elution of the mRNA.

34. The method of any one of claims 1-33, wherein the concentration of each ion pairing agent in the mobile phase is held constant during elution of the mRNA.

35. The method of any one of claims 1-34, wherein the concentration of one or more ion pairing agents in the mobile phase is not held constant during elution of the mRNA.

36. The method of any one of claims 1-35, wherein the eluting is gradient or isocratic with respect to the concentration of the organic solvent.

37. The method of any one of claims 1-36, wherein the method has a run time of between about 10 minutes and about 30 minutes.

38. The method of any one of claims 1-37, wherein a composition added to the column comprises the target mRNA in an amount ranging from about 0.05 mg/mL to about 1 mg/mL, optionally wherein the amount is 0.1 mg/mL.

39. The method of any one of claims 1-38, wherein the method further comprises repeating steps (i) through (iii) without an intervening step of regenerating the reverse phase chromatography column.

40. The method of any one of claims 1-39, wherein the method further comprises comparing the retention time of the target mRNA to the retention time of a reference nucleic acid, optionally wherein the reference nucleic acid is an unformulated mRNA, optionally wherein the comparing step comprises comparing an HPLC chromatogram of the identified nucleic acid with an HPLC chromatogram of the reference mRNA.

41. The method of any one of claims 1-40, wherein the method further comprises the step of isolating the target mRNA, optionally wherein the method is used to determine the potency of the target mRNA.

42. A method of quality control of a pharmaceutical composition comprising a target mRNA, the method comprising:
(i) identifying the target mRNA by the method of any one of claims 1-41;
(ii) comparing the separated mRNA with a reference mRNA; and
(iii) determining that the pharmaceutical composition comprises the target mRNA based on a comparison of the identified mRNA with the reference mRNA,
optionally wherein the comparing step comprises comparing a HPLC chromatogram of the identified mRNA with a HPLC chromatogram of the reference mRNA.
